(19) United States
(12) Patent Application Publication
(10) Pub. No.: US 2003/0060438 A1
(43) Pub. Date: Mar. 27, 2003

(54) Oligonucleotides and other modulators of the NK-1 receptor pathway and therapeutic uses thereof

(76) Inventors: James L. Henry, St. Hippolyte (CA); Catherine M. Cahill, Verdun, CA (US); Kiran Yashpal, Westmount, CA (US)

Correspondence Address: VENABLE, BAETJER, HOWARD AND CIVILETTI, LLP, P.O. BOX 34385, WASHINGTON, DC, 20043-9998, US

(21) Appl. No.: 09/930,503

(22) Filed: Aug. 16, 2001

Related U.S. Application Data
(60) Provisional application No. 60/226,086, filed on Aug. 18, 2000.

(51) Int. Cl. A61K 48/00

(52) U.S. Cl. 514/44

(57) ABSTRACT

Methods for administering oligonucleotides, nucleotide analogs, and non-nucleotide disruptor compounds to modulate the NK-1receptor biosynthetic pathway in humans and other mammals are provided. Oligonucleotides, and especially antisense oligonucleotides complementary to nucleic acids in the pathway that produces the NK-1 receptor, and those complementary to nucleic acids in pathways that regulate NK-1 receptor production and function, are useful, inter alia, to reduce pain, inflammation, and the undesirable effects of many diseases and conditions that involve NK-1 receptors. Non-nucleotide disrupter compounds and nucleotide analog compounds that act to modulate the NK-1receptor biosynthetic pathway to thereby regulate NK-1 receptor production and function, are similarly useful. The invention relates also to pharmaceutical preparations for humans and other mammals containing one or more oligonucleotides, nucleotide analogs, or non-nucleotide disrupter compounds.

### FIELD OF THE INVENTION

The present invention relates to the use of oligonucleotides, nucleotide analogs, and non-nucleotide disrupter compounds, to modulate the NK-1 receptor biosynthetic pathway in order to reduce the undesirable effects of many diseases and conditions.

### BACKGROUND OF THE INVENTION

Chronic pain and inflammation are associated with numerous disease states including inflammatory bowel disease, psoriasis, and arthritis. Although pain and inflammation can be functional in acute conditions, in chronic conditions they often serve no purpose and become debilitating. Pathological forms of pain may develop. For example, hyperalgesia is an enhanced pain response to painful stimulation; allodynia is a sensation of pain as a result of a neutral stimulus on normal skin, both of which can result from nerve injury or inflammation and other pathological states. Neuralgia and phantom limb pain are also often intractable. Effective treatment for such conditions has proven difficult. Peripherally acting analgesics and anti-inflammatories, such as aspirin, are often unable to control the pain associated with chronic conditions; centrally acting drugs such as the opiates are inappropriate for long term treatment since they can result in addiction and habituation. There is a significant clinical need for new methods of treating conditions such as chronic pain and inflammation.

### BRIEF DESCRIPTION OF THE INVENTION

In accordance with this and other objects, the present invention provides methods and means for treating any pathological condition that is characterized, at least partially, by involvement of the NK-1 receptor. Conditions that involve pain or inflammation are likely to involve the NK-1 receptor biosynthetic pathway, and related pathways that can modulate the production or function of NK-1 receptors. In general, the present method comprises the step of administering to a mammal in need thereof, a therapeutically effective amount of at least one compound, such as an oligonucleotide, an oligonucleotide analog, or a non-nucleotide disruptor compound that interferes with the function or production of NK-1 receptors. In embodiments of the invention utilizing one or more oligonucleotides or oligonucleotide analogs, that is, where the target of the administered compound is a nucleic acid, the target can be any nucleic acid, for example, DNA, RNA, tRNA, mRNA, and rRNA. Also, RNA in ribozymal form can be used to practice some embodiments of the invention. Depending upon the specific use and therapeutic context, ribozymes and aptamers can be tailored to target specific portions of the NK-1 receptor biosynthetic pathway.

Preferably, the administered oligonucleotide or oligonucleotide analog is one or more selected from oligonucleotides or analogs that are complementary to a portion of nucleic acid in the NK-1 receptor biosynthetic pathway to the extent that production of the NK-1 receptor protein is modulated, for example, by binding of the administered compound to a nucleic acid that is involved in the sequelae of events or signals that would stimulate production of the NK-1 receptor, or that would enable its effective functioning. In accordance with this aspect of the invention, preferably the oligonucleotide or analog is one or more selected from the group consisting of DNA antisense oligonucleotides, RNA antisense oligonucleotides, DNA sense oligonucleotides, RNA sense oligonucleotides, and ribozymes. Examples of preferred oligonucleotides are as described herein and include those identified in the sequence listings described herein. As one of skill in the art will appreciate, any oligonucleotide, oligonucleotide analog, or non-nucleotide disrupter compound that interferes with the function or production of NK-1 receptors is within the scope of the present invention. Oligonucleotides or analogs of the invention that may be most effective for practicing the invention include those that are complementary to at least a portion of a nucleic acid in the NK-1 receptor biosynthetic pathway.

The present invention is useful for modulating the effects of the NK-1 receptor biosynthetic pathway and modulating pathways in any organism that possesses NK-1 receptors. In some preferred embodiments of the invention, mammals, and especially humans, are suitable subjects. Of course, other mammals, such as cows, horses, cats, dogs, sheep, pigs and rodents, are suitable subjects for the present invention.

Also in accordance with the objects of the invention, pharmaceutical preparations are provided. Pharmaceutical preparations according to the invention comprise at least one oligonucleotide, oligonucleotide analog or a non-nucleotide disruptor. A disruptor is a compound that is not necessarily an oligonucleotide or oligonucleotide analog but still disrupts the NK-1 receptor biosynthetic pathway to the extent that the production or function of at least a portion of the NK-1 receptor is effected.

Compounds of the invention can be delivered in any manner consistent with their function and efficacy. For example, pharmaceutical preparations of the oligonucleotides, analogs, and non-nucleotide disrupter compounds of the invention can be provided in admixture with at least one pharmaceutically acceptable substance that assists or enhances the delivery or effectiveness of the compounds of the invention. Examples of such substances include, but are not limited to, excipients, penetration enhancers, stabilizers, absorption enhancers and carriers.

Delivery of compounds of the invention, and in accordance with the methods described herein, can be effected in any manner that results in delivery of the compounds of the invention such that interference with the NK-1 receptor biosynthesis pathway is accomplished. For example, compounds of the invention such as oligonucleotides, oligonucleotide analogs and non-nucleotide disruptors, can be administered by intrathecal infusion to the spinal canal, by intravenous infusion, or by one or more routes such as, but not limited to, oral, buccal, mucosal, parenteral, rectal, sub-cutaneous, transdermal, intravaginal, nasal, nasal inhalation, pulmonary inhalation, iontophoresis through the skin, iontophoresis through mucosal or buccal membranes, dermal patch, epidural, intracranial, intrapharyngeal, sublingual, intra-articular, intramuscular, and subcutaneous.

As one of skill in the art will comprehend, appropriate dosages of compounds according to the various embodiments of the invention can vary widely depending, inter alia, upon the type of disease or condition to be treated, the route of treatment, the subject mammal, and the sequelae of symptoms and compounds involved. Dosages range greatly, for example, between 10 nanomoles and 900 micrograms per kilogram of body weight of the mammal. Some typical ranges for the amount of the oligonucleotide, oligonucleotide analog or non-nucleotide NK-1 disruptor compound to be administered include preferably from 15 to 30 nanomoles per kilogram of body weight of the mammal, in the range of from 20 to 25 nanomoles per kilogram of body weight of the mammal, or from 15 to 30 nanomoles per kilogram of body weight of the mammal. In some preferred embodiments, the compound is administered in amounts from 50 to 600 micrograms per kilogram or from 200 to 400 micrograms per kilogram of body weight of the mammal, or from 250 to 350 micrograms per kilogram of body weight of the mammal.

The methods and pharmaceutical compositions of the present invention can be used to treat any disease or condition that involves the NK-1 receptor biosynthetic pathway, or the NK-1 receptor modulation pathways. Examples of such diseases and conditions include, among others, dermatological disorders, immune disorders, autoimmune disorders, cardiovascular disorders, neuropathic disorders, vascular disorders, gut inflammation, arthritis, airway disorders, central aspects of chronic or acute pain, peripheral aspects of chronic or acute pain, psychiatric disorders, and central nervous system disorders. Among these are urticaria, migraine, anxiety, psychosis, and schizophrenia. Also included among the many diseases and conditions that can be treated or prevented by the pharmaceutical compositions and methods of the present invention are pain and inflammation of any etiology so long as involvement of the NK-1 receptor is present.

In accordance with these aspects of the invention, methods are provided comprising, administering to a mammal in need thereof, a therapeutically effective amount of at least one compound that interferes with the function or production of NK-1 receptors. Preferably, the compound is at least one selected from the group comprising oligonucleotides, nucleotide analogs, non-nucleotide disruptor compounds, and the interference with the function or production of the NK-1 receptors involves action of the compound on at least one nucleic acid in the NK-1receptor biosynthetic pathways selected from the group including DNA, RNA, tRNA, mRNA and rRNA. The oligonucleotide may be RNA in the form of one or more ribozymes.

Preferably, the oligonucleotide or nucleotide analog can be one or more selected from oligonucleotides that are complementary to nucleic acid in the NK-1 receptor biosynthetic pathway, and are one or more selected from the group comprising DNA antisense oligonucleotides, RNA antisense oligonucleotides, DNA sense oligonucleotides, RNA sense oligonucleotides, aptamers and ribozymes. More specifically, preferred oligonucleotides include one or more selected from the group described in the Sequence Listings provided herewith as well as any others that function to modulate the NK-1 biosynthetic pathway. As one of skill in the art will comprehend, any oligonucleotides that are complementary to nucleic acids in the NK-1 receptor biosynthesis pathway are particularly desirable for practicing some embodiments of the invention. Thus, oligonucleotides complementary to nucleic acids included in the Sequence Listings described herein are also suitable for practicing the invention.

Methods, kits and pharmaceutical compositions of the present invention are useful for treating, diagnosing or preventing different types of pain such as, but not limited to, those characterized as peripheral pain, neuropathic pain, chronic pain, acute pain, pain relating to psychiatric disorders, and pain relating to central nervous system disorders. Furthermore, the types of pain may be characterized by hyperalgesia, allodynia, neuralgia, or dysesthesia. Methods and pharmaceutical compositions of the present invention are applicable also to treating, diagnosing or preventing different types of inflammatory conditions, including but not limited to, wherein the inflammation is characterized as peripheral inflammation, chronic inflammation, acute inflammation, neuropathic inflammation, inflammation relating to psychiatric disorders, or inflammation relating to central nervous system disorders. Inflammation for which the present invention is suitable include those that may be characterized as arising from, or associated with, hyperalgesia, allodynia, neuralgia, or dysesthesia.

Non-nucleotide disruptors according to the invention may include one or more methylation compounds, de-methylation compounds, mutagens, intercalation compounds, gyrases, ligases, and methylases. In accordance with the present methods and compositions, the non-nucleotide disruptor or disruptors can act directly or indirectly upon nucleic acid in the NK-1 receptor biosynthetic or modulating pathways, or can act on proteins or other molecules involved in the NK-1 biosynthetic pathway, or on the NK-1 protein itself. The present invention includes also kits and methods for treating, diagnosing or preventing any condition that is characterized at least partially by activation of the NK-1 receptor biosynthetic and modulating pathways. The kits include, inter alia, a therapeutically effective amount of at least one compound that interferes with the function or production of NK-1 receptors as described herein. In some embodiments, a kit according to the pharmaceutical compound is provided in admixture with at least one pharmaceutically acceptable substance from the group consisting of excipients, penetration enhancers, stabilizers, absorption enhancers and carrier compounds. In accordance with the several objects of the invention, the compound may be one or more disruptors that are not a nucleic acid or nucleic acid analog, and are selected from the group consisting of methylation compounds, de-methylation compounds, antibodies, mutagens, intercalation compounds, gyrases, ligases, and methylases. In embodiments of kits that are directed toward nucleic acids in the NK-1 biosynthetic pathway and related modulating pathways, appropriate oligonucleotides are provided. Kits of the invention can be designed to target any pathological condition that involves the NK-1receptor biosynthetic pathway as described herein.

### BRIEF DESCRIPTION OF THE FIGURES

FIG. 1. Shows a time course of substance P induced hyperalgesia, which is attenuated by intrathecal oligonucleotide administration.

FIG. 2. Shows reduction of the response to a painful stimulus, capsaicin, which is attenuated by intrathecal oligonucleotide administration.

FIG. 3. Shows reduction of the response to a painful stimulus, formalin, which is attenuated by intrathecal oligonucleotide administration.

FIG. 4. Shows no change in of the response to a painful stimulus, formalin, after low dose intrathecal oligonucleotide.

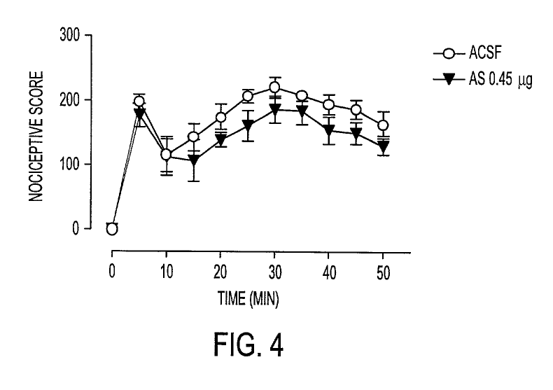

FIG. 5. Shows reduction of the response to chronic neuropathic pain, which is attenuated by intrathecal oligonucleotide administration, but no effect of sensory threshold on the non-injured side.

FIG. 6. Shows a comparison of contralateral versus ipsilateral response to a non-painful stimulus after intrathecal oligonucleotide stimulus, in naive rats.

FIG. 7. Shows a comparison of contralateral versus ipsilateral response to a painful stimulus after nerve injury in a model of chronic neuropathic pain, after delivery of intrathecal oligonucleotides to a different cord segment.

FIG. 8. Shows a comparison of contralateral versus ipsilateral response to a normally non-painful stimulus after nerve injury in a model of chronic neuropathic pain

FIG. 9. Shows a comparison of contralateral versus ipsilateral response to a normally non-painful stimulus after nerve injury in a model of chronic neuropathic pain

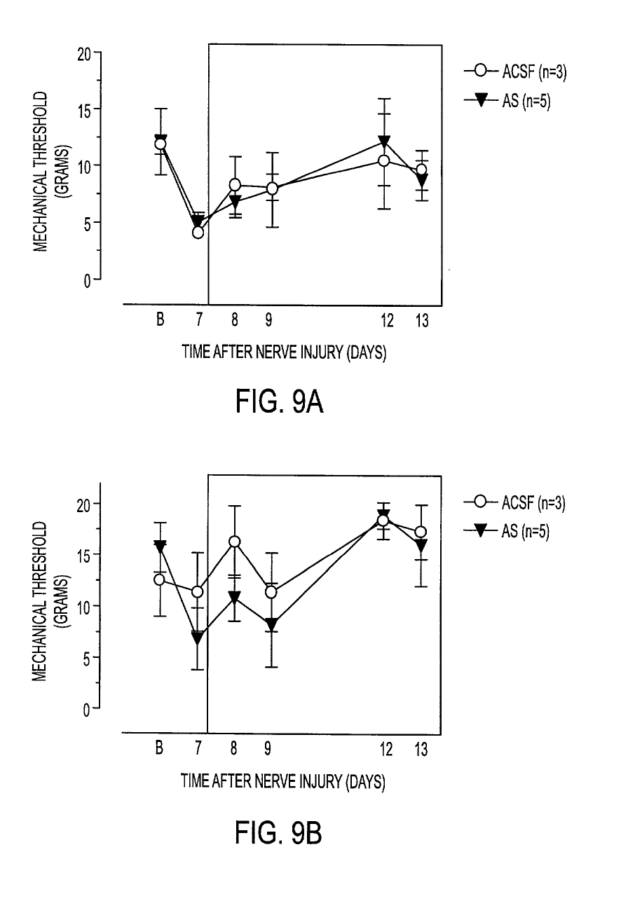

FIG. 10. Shows a Western Blot, comparing labeling by artificial cerebrospinal fluid(ACSF), missense and antisense probes of NK-1 receptor.

FIG. 11 Shows attenuation of inflammation by intrathecal antisense (AS) oligonucleotides in an animal model of arthritis.

FIG. 12. Shows reduction of the response to a painful stimulus, formalin, which is attenuated by systemic oligonucleotide administration.

FIG. 13. Shows attenuation of inflammation by systemic administration of AS oligonucleotides in an animal model of arthritis.

FIG. 14. Shows increased mobility in an animal model of arthritis after treatment with systemic administration of AS oligonucleotide.

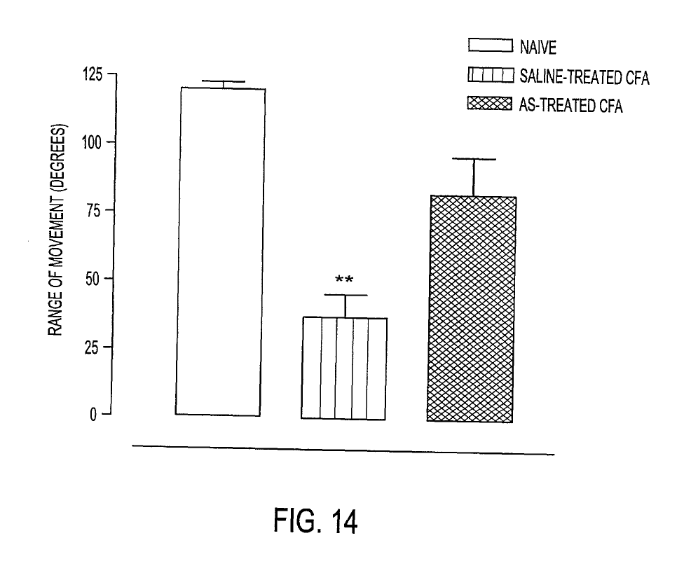

FIG. 15. Shows reduced breakdown of collagen in animal model of arthritis, after treatment with AS oligonucleotide by peripheral administration.

FIG. 16. Shows reduced plasma extravasation in rat colon treated with peripheral administration AS oligonucleotides.

FIG. 17. Shows reduced plasma extravagation to skin in rats treated peripherally with AS oligonucleotides.

FIG. 18. Shows immuno-histochemical localization of NK-1 receptor in the rat lumbar spinal cord.

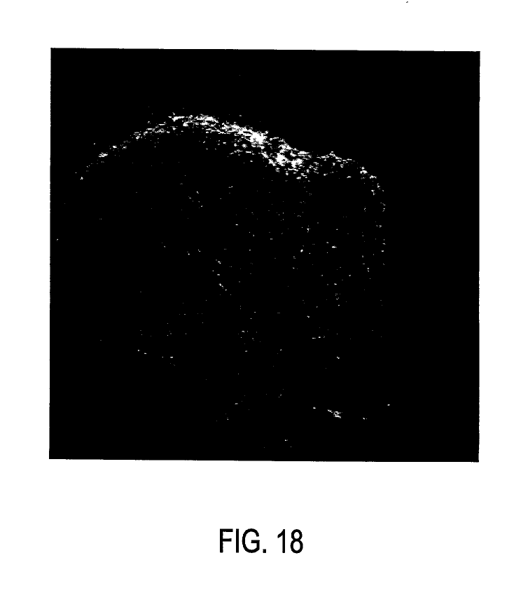

### DETAILED DESCRIPTION OF THE INVENTION

The mechanisms of chronic pain and inflammation have begun to be elucidated. These can be associated with a wide range of conditions. Inflammation is a protective response of tissues affected by disease or injury, and characterized by redness, localized heat, swelling, pain, and possibly impaired function of the affected part. Recently a common mechanism for pain and inflammation has been identified in the substance P/tachykinin: neurokinin receptor 1 (NK-1) system. Substance P, C₆₃H₉₈N₁₈O₁₃S, is a neurotransmitter composed of amino acids, (sequence: Arg-Pro-Lys-Pro-Gly-Gln-Phe-Phe-Gly-Leu-Met-NH₂). It is a member of the tachykinin family of peptide hormones that is present in nerve cells and in certain endocrine and immune cells. It induces contraction of intestinal smooth muscle and vasodilation; in the central nervous system, it acts as a neurotransmitter in the pain pathway.

A subset of neurons with cell bodies in the dorsal root ganglia use substance P as a transmitter and are activated by certain painful and/or inflammatory stimuli. Following tissue damage or in some disease states or conditions kinins are produced in the plasma and tissue that activate substance P containing cells. The central process of these neurons activate NK-1 receptors on neurons in the dorsal spinal cord. The peripheral process of these neurons is involved in the inflammatory response. Chronic pain and chronic inflammatory diseases are associated with increased numbers NK-1 receptors. Peripherally this system has been implicated in chronic pain, chronic inflammation, and neuronal, intestinal, bone, vascular, and skin disorders. Within the central nervous system, this system has been implicated in anxiety, psychosis, schizophrenia, and glioma. Previous investigators have attempted to interfere in the system by means of NK-1 receptor antagonists, see for example, Dorn, et al. U.S. Pat. No. 5,716,942 and Horwell et al. U.S. Pat. No. 5,981,755. NK-1 receptor antagonists are effective to block pain and inflammation in animal studies but have not been effective as therapeutic agents, particularly in treating human subjects.

The present invention is based on the insight that disruption of the pathway starting with the gene for the NK-1 receptor and ending with production of the protein which migrates to the surface of cells to become a functional NK-1 receptor. This invention is an effective method of reducing activation of the NK-1 receptor both centrally and peripherally. For example, administration of antisense oligonucleotides complementary to nucleic acids involved in the NK-1 receptor can prevent or reduce pain and inflammation. The gene for NK-1 receptor has been sequenced (see Fong and Strader, U.S. Pat. Nos. 5,484,886 and 5,525,712), as has mRNA for the NK-1 receptor, see SEQ ID NO 1-8, for nucleic acid and peptide sequences. This allows construction of antisense oligonucleotides, see below. However, a recent study of acute pain, using antisense oligonucleotides for NK-1 receptors administered intrathecally, found only minor effects except during co-administration of substance P with the oligonucleotides. Hua, X. et al., J. Neurochem., Vol. 70: 688-698 (1998). The lack of responsiveness to oligonucleotides, found by Hua et al., may have been due to the fact that NK-1 receptors normally turn over at a slow rate, and thus, interfering with NK-1 receptor production may have little short-term effect on receptor numbers. In an unexpected contrast to the findings of Hua et al., the present inventors have found that oligonucleotides, and especially antisense oligonucleotides, can be used effectively to treat chronic conditions and other pathological states without the co-administration of substance P. In such pathological states, the activation of NK-1 receptors is already high and turnover rates are commensurately rapid. Treatment with antisense oligonucleotides appears to reduce the number of activated receptors while not reducing the number of quiescent NK-1 receptors. Thus, the present invention targets NK-1 receptors that are active because of an existing condition to thereby ameliorate chronic pain and inflammation and disease conditions associated therewith. Receptors not chronically stimulated will be less affected, reducing side effects of treatment.

NK-1 receptor related disorders, diseases, or pathological conditions include but are not limited to: respiratory conditions (e.g. asthma, allergic rhinitis, cystic fibrosis (CF)), ophthalmic conditions (e.g. conjunctivitis), cutaneous/dermatologic conditions (e.g. allergic dermatitis, dermatitis by contact, psoriasis rosacea, sensitivity to environmental stimuli), intestinal conditions (e.g. ulcerative colitis, Crohn's disease, inflammatory bowel diseases, inflammations of the gut), cardiovascular conditions (stroke, cardiac ischemia, peripheral vascular disease, migraine); airway disruption (CF, asthma, bronchitis), chronic gastrointestinal tract inflammation, pain, central nervous system disorders such as anxiety, psychosis, depression, and neuro-degenerative disorders, inflammatory diseases such as rheumatoid arthritis and inflammatory bowel diseases, as well as pain in any of the aforesaid conditions, including migraine. Also included is pain itself such as chronic pain, neurogenic pain, neuropathic pain or pain that follows injury or pressure to nerves, chronic pain, phantom limb pain, and pain characterized by hyperalgesia, allodynia, neuralgia, and dysesthesia.

Other disorders or diseases include, but are not limited to, cardiovascular pathologies, including stroke, cardiac ischemia, peripheral vascular disease and migraine, edema, such as edema caused by thermal injury, chronic inflammatory diseases such as rheumatoid arthritis, asthma/bronchial hyper-reactivity and other respiratory diseases including allergic rhinitis, inflammatory diseases of the gut, including irritable bowel syndrome, ocular injury and ocular inflammatory diseases, proliferative vitreo-retinopathy, irritable bowel syndrome, disorders of bladder function including cystitis and bladder detrusor hyperreflexia, demyelinating diseases such as multiple sclerosis and amyotrophic lateral sclerosis, asthmatic disease, small cell carcinomas, and particularly small cell lung cancer, depression, dysthymic disorders, chronic obstructive airways disease, hypersensitivity disorders such as allergies and poison ivy, vasospastic diseases such as angina and Reynauld's disease, fibrosing and collagen diseases such as scleroderma and eosinophilic fascioliasis, reflex sympathetic dystrophy such as shoulder/hand syndrome, addiction disorders such as alcoholism, stress related somatic disorders, neuropathy, neuralgia, tendinitis, phantom limb pain, disorder related to immune enhancement or suppression such as systemic lupus erythmatosis conjunctivitis, vernal conjunctivitis, contact dermatitis, atopic dermatitis, urticaria, and other eczematoid dermatitis; central nervous system disorders such as anxiety, depression, psychosis and schizophrenia; neuro-degenerative disorders such as AIDS related dementia, senile dementia of the Alzheimer type, Alzheimer's disease and Down's syndrome; Huntington's Chorea, epilepsy, and other neuro-pathological disorders such as peripheral neuropathy, inflammatory diseases such as inflammatory bowel disease, irritable bowel syndrome, psoriasis, fibrositis, ocular inflammation, osteoarthritis and rheumatoid arthritis; allergies such as eczema and rhinitis; hypersensitivity disorders such as poison ivy; ophthalmic diseases such as conjunctivitis, vernal conjunctivitis, dry eye syndrome, and the like; cutaneous diseases such as contact dermatitis, atopic dermatitis, urticaria, and other eczematoid dermatitis; edema, such as edema caused by thermal injury; stress related somatic disorders; reflex sympathetic dystrophy such as shoulder/hand syndrome; dysthymic disorders; neuropathy, such as diabetic or peripheral neuropathy and chemotherapy-induced nemopathy; post-herpetic and other neuralgias; asthma; osteoarthritis; rheumatoid arthritis; and migraine.

The subjects to be treated, or whose tissue may be used in accordance with the present invention, may be any organism possessing NK-1 receptors, such as a an animal, especially a mammal or, more specifically, a human, non-human primate, horse, pig, cow, sheep, rabbit, dog, cat, monkey, or rodent. In one preferred embodiment of the present invention, the subject is a human. A pathological condition is any disease, dysfunction, malady, or disorder that may cause or relate to a condition causing discomfort, stiffness, or pain to the subject or which interferes with a normal or usual function of the subject's body. Without intending to limit the invention, applicants theorize that by controlling or moderating the relative population of NK-1 receptors in a target tissue, the pathological condition, or at least its symptoms, can be relieved or ameliorated. Thus, by administering oligonucleotides complementary to nucleic acids in the cellular pathways involved in producing or modulating NK-1 receptors, the relative number of receptors can be reduced in tissues where there are high levels of NK-1 activity. As a corollary to this theory, in tissues at low activity levels, the present invention would have little effect because of the stability and low turnover rate of NK-1 receptors. Regardless of the underlying mechanism or mechanisms by which the present invention works, applicants have discovered that administering the appropriate oligonucleotides, nucleotide analogs, or non-nucleotide disruptors as described herein, works to reduce NK-1-mediated phenomena such as inflammation or pain.

In accordance with the present invention, oligonucleotides may comprise nucleotide sequences sufficient in identity and number to effect specific hybridization with a particular nucleic acid. One category of such oligonucleotides are commonly described as “antisense.” Antisense oligonucleotides are commonly used as research reagents, diagnostic aids, and therapeutic agents. In the context of the invention, any part of a pathway that affects the production or function of the NK-1 receptor can be a target for an oligonucleotide, an oligonucleotide analog or a non-nucleotide disrupter. The term “disrupter” is understood to include any molecule, or group of molecules which disturb, perturb, disrupt or interfere with any process, particularly a process leading to the production of a protein from DNA. The disruption or interference should be sufficient to reduce the amount of the protein or to diminish the activity of the protein, or to interfere with its use or utility. In the context of the present invention disrupter would include any pharmaceutically acceptable non-oligo compound that interferes with the function or production of NK-1 receptors. Thus, targets for the methods and pharmaceutical preparations of the present invention include not only nucleic acids in the pathway from the gene for the NK-1 receptor to the resultant NK-1 receptor protein(s) but nucleic acids in the pathways for effectors for the NK-1 receptor gene such as the genes for promoters, modulators, and repressors of the NK-1 gene and gene product or any fragment segment or partion thereof.

The oligonucleotides of the present invention include those known as ribozymes, or RNA molecules with enzymatic activity. Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Several natural ribozyme motifs have been identified based on their physical structure, biological and biochemical properties. In general natural ribozymes are categorized according to their specialized catalytic properties:

1. Hammerhead, hairpin and hepatitis delta virus (HDV) have the ability to self-cleave a particular phosphodiester bond.

2. Group I and II intron ribozymes can self-splice, and can cleave and ligate phosphodiester bonds.

3. Ribonuclease P ribozyme cleaves a phosphodiester bond in a variety of cellular tRNA precursors.

The enzymatic nature of a ribozyme can be advantageous over other technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action. Thus, the specificity of action of a ribozyme is greater than that of an antisense oligonucleotide binding the same RNA site. U.S. Pat. Nos. 5,591,610, 6,132,967, and 6,265,167 provide methods of constructing and targeting ribozymes.

Thus, the present invention encompasses any oligonucleotide, or nucleotide analog, that sufficiently hybridizes to, or is sufficiently complementary to, a nucleic acid involved in the NK-1 receptor biosynthetic pathway to the extent that interference with production or function of the NK-1 receptor protein is effected. The methods and preparations of the present invention include ribozymes, sense and antisense DNA and RNA, which hybridize to any portion of genes involved in the NK-1receptor biosynthetic pathway, genomic DNA, mRNA, tRNA, and rRNA where such hybridization occurs to the extent necessary that interference with the production or function of active receptors on the surface of target cells in a target tissue is accomplished. Similarly, non-nucleotide disruptors include any compound that accomplishes the same result.

Oligonucleotides and nucleotide analogs of the invention also include any that can hybridize to a nucleic acid in the pathway leading to the production of active NK-1 receptors. Preferred are nucleotides listed below with SEQ ID NO's 9 to 59. Most preferred is an antisense nucleotide sequence that hybridizes to a portion of the mRNA that includes the initiation codon for translation of bases 575-577 on the mRNA sequence for the receptor, SEQ ID NO 9 to 21.

The present invention comprises compounds and methods for inhibiting activation or production of NK-1 receptors using the oligonucleotides, nucleotide analogs and disruptors of the invention. Methods are also provided of treating conditions in which abnormal or excessive substance P-mediated inflammation or pain occurs. These methods employ the oligonucleotides of the invention and are believed to be useful both therapeutically and as clinical research and diagnostic tools. The compounds of the present invention may also be used for research purposes. Thus, the specific hybridization exhibited by the oligonucleotides and nucleotide analogs, as well as the non-nucleotide disruptors, of the present invention may be used for assays, purifications, cellular product preparations and in other methodologies which may be appreciated by persons of ordinary skill in the art.

The present invention employs molecules such as oligonucleotides or nucleotide analogs for use in modulation of the function of DNA or messenger RNA (mRNA) encoding a protein the modulation of which is desired, and ultimately to regulate the amount of such a protein. Preferred is antisense modulation. Hybridization of an antisense oligonucleotide with its mRNA target interferes with the normal role of mRNA and causes a modulation of its function in cells. The functions of mRNA to be interfered with include all vital functions such as translocation of the RNA to the site for protein translation, actual translation of protein from the RNA, splicing of the RNA to yield one or more mRNA species, and possibly even independent catalytic activity which may be engaged in by the RNA. The overall effect of such interference with mRNA function is modulation of the expression of a protein, wherein “modulation” means either an increase (stimulation) or a decrease (inhibition) in the expression of the protein. In the context of the present invention, inhibition is the preferred form of modulation of gene expression. As one of skill in the art can appreciate, appropriate ribozymes are also within the scope of the present invention. The present invention pertains to a method for regulating gene expression in the pathway leading to the production of the NK-1 receptor through inhibition of gene expression by nuclear antisense RNA. The for example for example, U.S. Pat. No. 6,265,167 provides an efficient means for introducing, expressing and accumulating the antisense RNA in the nucleus. The antisense RNA hybridizes to the sense mRNA in the nucleus, thereby preventing both processing and cytoplasmic transport. The construct comprises a promoter, an origin of replication, antisense sequences, and a cis- or trans-ribozyme which generates 3′-ends independently of the polyadenylation machinery and thereby inhibits the transport of the RNA molecule to the cytoplasm. The construct may also comprise a histone stem-loop structure that assists in stabilizing the transcripts against exonucleolytic degradation.

This method provides certain advantages over other prior cytoplasmic antisense technology. First, this invention closely mimics the system of naturally-occurring antisense regulation seen in a variety of organisms, indicating that it is a natural means for studying antisense regulation of gene expression. Also, this invention solves at least one problem created by cytoplasmic antisense RNA, namely the activation of interferon by double stranded RNAs. There is no indication that nuclear antisense RNA causes interferon activation, and therefore there is less risk of adverse effects on the cell.

Once introduced into the cell nucleus, the construct begins expressing the antisense sequences following the promoter. The construct contains none of the usual transcription termination sequences and is inserted to cleave the transcript without normal polyadenylation. This variation prohibits transportation of the antisense sequences from the nucleus to the cytoplasm. As the antisense sequences accumulate in the nucleus, they hybridize to their complementary sense RNA transcripts. It is believed that the formation of these hybrids prevents processing and cytoplasmic transport of the RNA, as these hybrids are shown to remain in the nucleus and are eventually degraded. By hybridizing to a targeted gene, the antisense transcripts can regulate and inhibit expression of that gene. This function has utility in both therapeutic and research applications.

Other molecules which can interfere with the pathway from DNA to production of functional NK-1 receptor are included in the present invention. “Aptamers” are nucleic acid molecules which are constructed and selected for their ability to bind to proteins. Briefly the protein sequence of the NK-1 receptor can be used to construct complementary nucleic acid sequences, which are repeatedly selected for their ability to bind to the NK-1 receptor. The biological function of nucleic acids was thought to be reserved to base pairing with other nucleic acids or to interactions with proteins that had evolved to bind nucleic acids. It is now known that nucleic acids possess structural as well as functional complexity called aptamers.

Aptamers have been identified by a procedure of cycled amplification and selection steps referred to as SELEX (systematic evolution of ligands by exponential enrichment). The targets of aptamers range from proteins known to bind nucleic acid, to proteins not thought to associate with nucleic acids in vivo, to small molecules. With nucleic acid binding targets, a perfect target sequence can be found to investigate the genome for undiscovered interaction sites, or the molecular interactions involved can be characterized by comparing different aptamers with similar binding affinity. Introduction of such aptomers in a system where NK-1 receptors are rapidly turning over could prevent receptors from being replaced.

Similarly antibodies to nucleic acids in the pathway leading to the NK-1 receptor. Antibodies, particularly monoclonal antibodies, can be constructed by any convention means known to those of skill in the art. Such antibodies could be constructed to react with one or more of the nucleic acids in the pathway and could be used to block or reduce production of the receptor protein or otherwise produce a therapeutic response. Such antibodies also have utility as diagnostic and research reagents, and in purification and quantitation of components of the pathway.

It is preferred to target specific genes for antisense attack or other disruption. “Targeting” an oligonucleotide to the associated nucleic acid, in the context of this invention, is a multi-step process. The process begins with the identification of a nucleic acid sequence whose function is to be modulated. In the present invention the gene to be targeted is the NK-1 receptor gene. The targeting process also includes determination of a site or sites within this gene for the oligonucleotide interaction to occur such that the desired effect, either detection or modulation of expression of the protein will result. Once the target site or sites have been identified, oligonucleotides are chosen which are sufficiently complementary to the target, i.e., hybridize sufficiently well and with sufficient specificity to give the desired effect.

Generally, there are five regions of a gene that may be targeted for antisense modulation: the 5′ untranslated region (hereinafter, the “5′-UTR”), the translation initiation codon region (hereinafter, the “tTR”), the open reading frame (hereinafter, the “ORF”) the translation termination codon region (hereinafter, the “tTR”) and the 3′ untranslated region (hereinafter, the “3′-UTR”). As is known in the art, these regions are arranged in a typical messenger RNA molecule in the following order (left to right, 5′ to 3′): 5′-UTR, tIR, ORF, tTR, 3′-UTR. As is known in the art, although some eukaryotic transcripts are directly translated, many ORFs contain one or more sequences, known as “introns,” which are excised from a transcript before it is translated; the expressed (unexcised) portions of the ORF are referred to as “exons” (Alberts et al., Molecular Biology of the Cell, 1983, Garland Publishing Inc., New York, pp. 411-415). Furthermore, because many eukaryotic ORF's are a thousand nucleotides or more in length, it is often convenient to subdivide the ORF into, e.g., the 5′ ORF region, the central ORF region, and the 3′ ORF region. In some instances, an ORF contains one or more sites that may he targeted due to some functional significance in vivo. Examples of the latter types of sites include intragenic stem-loop structures (see, e.g., U.S. Pat. No. 5,512,438) and, in unprocessed mRNA molecules, intron/exon splice sites. The present invention encompassed a range of antisense oligonucleotides which target NK-1 receptor DNA in areas particularly in the area of and including the initiation coding site for transcription of other oligonucleotides.

An example for antisense targeting of a specific site is given below wherein antisense oligonucleotides were constructed to target the initiation codon for transcription (ICT). Thirteen sequences were constructed to form different lengths oligonucleotides which would be complementary around the ICT. Many such sequences could be constructed, which are complementary to adjacent or over lapping sequences or DNA or RNA. One of skill in the art will recognize that antisense oligonucleotides can be slightly longer or shorter without interfering with their ability to interfere with biosynthesis of proteins.

TABLE 1
ANTISENSE Sequences

| | |
|---|---|
| SEQ ID NO 9 | 5′ GAC GTT ATC CAT TTT GGG GCA 3′ |
| SEQ ID NO 10 | 5′ GAC GTT ATC CAT TTT GGG GC 3′ |
| SEQ ID NO 11 | 5′ GAC GTT ATC CAT TTT GGG G 3′ |
| SEQ ID NO 12 | 5′ GAC GTT ATC CAT TTT GGG 3′ |
| SEQ ID NO 13 | 5′ GAC GTT ATC CAT TTT GG 3′ |
| SEQ ID NO 14 | 5′ GAC GTT ATC CAT TTT G 3′ |
| SEQ ID NO 15 | 5′ GAC GTT ATC CAT TTT 3′ |
| SEQ ID NO 16 | 5′ AC GTT ATC CAT TTT GGG GCA 3′ |
| SEQ ID NO 17 | 5′ C GTT ATC CAT TTT GGG GCA 3′ |
| SEQ ID NO 18 | 5′ GTT ATC CAT TTT GGG GCA 3′ |
| SEQ ID NO 19 | 5′ TT ATC CAT TTT GGG GCA 3′ |
| SEQ ID NO 20 | 5′ T ATC CAT TTT GGG GCA 3′ |
| SEQ ID NO 21 | 5′ ATC CAT TTT GGG GCA 3′ |

Within the context of the present invention, one preferred intragenic site is the region encompassing the translation initiation codon of the open reading frame (ORF) of the gene. Because, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Furthermore, 5′-UUU functions as a translation initiation codon in vitro. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions, in order to generate related polypeptides having different amino terminal sequences. In the context of the invention, “start codon” and “translation initiation codon” refer to the codon or codons that are used in vivo to initiate translation of an mRNA molecule transcribed from a gene encoding a NK-1 receptor protein, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination region” refer to a portion of such an mRNA or gene that encompasses from about 25 to about 50 contiguous nucleotides in either direction (i.e., 5′ or 3′) from a translation termination codon.

The remainder of the Detailed Description relates in more detail the (1) Oligonucleotides of the Invention and their (2) Bioequivalents, (3) Utility, (4) Pharmaceutical Compositions and (5) Means of Administration

1. Oligonucleotides of the Invention

In one preferred embodiment, the present invention employs oligonucleotides for use in modulation of one or more NK-1 receptor proteins. In the context of this invention, the term “oligonucleotide” refers to an oligomer or polymer of ribonucleic acid or deoxyribonucleic acid. This term includes oligonucleotides composed of naturally occurring nucleobases, sugars and covalent inter-sugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

An oligonucleotide is a polymer of a repeating unit generically known as a nucleotide. The oligonucleotides in accordance with this invention preferably comprise from about 8 to about 30 nucleotides. An unmodified (naturally occurring) nucleotide has three components: (1) a nitrogen-containing heterocyclic base linked by one of its nitrogen atoms to (2) a 5-pentofuranosyl sugar and (3) a phosphate esterified to one of the 5′ or 3′ carbon atoms of the sugar. When incorporated into an oligonucleotide chain, the phosphate of a first nucleotide is also esterified to an adjacent sugar of a second, adjacent nucleotide via a 3′-5′ phosphate linkage. The “backbone” of an unmodified oligonucleotide consists of (2) and (3), that is, sugars linked together by phospho-diester linkages between the 5′ carbon of the sugar of a first nucleotide and the 3′ carbon of a second, adjacent nucleotide. A “nucleoside” is the combination of (1) a nucleobase and (2) a sugar in the absence of (3) a phosphate moiety. The backbone of an oligonucleotide positions a series of bases in a specific order; the written representation of this series of bases, which is conventionally written in 5′ to 3′ order, is known as a nucleotide sequence. Any type of novel or non-natural backbone structure can also be used in the present invention including but not restricted to novel backbone structures such as a phosphorothioate backbone and morpholino structures.

Oligonucleotides may comprise nucleotide sequences sufficient in identity and number to effect specific hybridization with a particular nucleic acid. Such oligonucleotides which specifically hybridize to a portion of the sense strand of a gene are commonly described as “antisense.” In the context of the invention, “hybridization” means hydrogen bonding, which may be Watson-Crick, Hoogsteen or reversed Hoogsteen hydrogen bonding, between complementary nucleotides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complimentarity or precise pairing such that stable and specific binding occurs between the oligonucleotide and the DNA or RNA target. An oligonucleotide is specifically hybridizable to its target sequence due to the formation of base pairs between specific partner nucleobases in the interior of a nucleic acid duplex. Among the naturally occurring nucleobases, guanine (G) binds to cytosine (C), and adenine (A) binds to thymine (T) or uracil (U). In addition to the equivalency of U (RNA) and T (DNA) as partners for A, other naturally occurring nucleobase equivalents are known, including 5-methylcytosine, 5-hydroxymethylcytosine (HMC), glycosyl HMC and gentiobiosyl HMC (C equivalents), and 5-hydroxymethyluracil (U equivalent). Furthermore, synthetic nucleobases which retain partner specificity are known in the art and include, for example, 7-deaza-Guanine, which retains partner specificity for C. Thus, an oligonucleotide's capacity to specifically hybridize with its target sequence will not be altered by any chemical modification to a nucleobase in the nucleotide sequence of the oligonucleotide which does not significantly effect its specificity for the partner nucleobase in the target oligonucleotide.

One of skill in the art will recognize that, within the contest of the invention, “sense” and “antisense” can be reletive terms, and a oligonucleotide that is antisense to a DNA molecule may be sense to a RNA molecule. Thus the invention encompasses oligonucleotides that are complementary to both DNA and RNA. The following table lists oligonucleotide which are complementary to nucleic acids in the NK-1 receptor biosynthesis pathway.

TABLE 2

| Sense | | Antisense | |
|---|---|---|---|
| ttc cac atc ttc ttc ctc ct | (SEQ ID NO 22) | agg agg aag aag atg tgg | (SEQ ID NO 41) |
| | | aa | |
| tga tga ttg tcg tgg tgt gca | (SEQ ID NO 23) | tgc aca cca cga caa tca | (SEQ ID NO 42) |
| | | tca | |
| gca agt ctc tgc caa gcg | (SEQ ID NO 24) | ttg cgc ttg gca gag act | (SEQ ID NO 43) |
| caa | | tgc | |
| ttg atg tag ggc agg agg aa | (SEQ ID NO 25) | ttc ctc ctg ccc tac atc aa | (SEQ ID NO 44) |
| tgc aca cca cga caa tca tca | (SEQ ID NO 26) | tga tga ttg tcg tgg tgt | (SEQ ID NO 45) |
| | | gca | |
| cat agt gtg att ccc act ac | (SEQ ID NO 27) | gta gtg gga atc aca cta | (SEQ ID NO 46) |
| | | tg | |
| atg cat agc caa tca cca gca | (SEQ ID NO 28) | tgc tgg tga ttg gct atg | (SEQ ID NO 47) |
| | | cat | |
| act ttg gtg gct gtg gct ga | (SEQ ID NO 29) | tca gcc aca gcc acc aaa | (SEQ ID NO 48) |
| | | gt | |
| gga tgt atg atg gcc atg ta | (SEQ ID NO 30) | tac atg gcc atc ata cat cc | (SEQ ID NO 49) |
| cat gga gta gat act ggc | (SEQ ID NO 31) | ttc gcc agt atc tac tcc | (SEQ ID NO 50) |
| gaa | | atg | |
| gaa gaa gtt gtg gaa ctt gca | (SEQ ID NO 32) | tgc aag ttc cac aac ttc | (SEQ ID NO 51) |
| | | ttc | |
| gta gac ctg ctg gat aaa ctt | (SEQ ID NO 33) | aag ttt atc cag cag gtc | (SEQ ID NO 52) |
| | | tac | |
| aca gta gat gat ggg gtt gta | (SEQ ID NO 34) | atg tac aac ccc atc atc | (SEQ ID NO 53) |
| cat | | tac | |
| gtg tac aga tag tag gct t | (SEQ ID NO 35) | aag cct act atc tgt aca c | (SEQ ID NO 54) |
| cct cct gtc tgg ctt tag aa | (SEQ ID NO 36) | ttc taa agc cag aca gga | (SEQ ID NO 55) |
| | | gg | |
| aac cca tac tga ccc ttt t | (SEQ ID NO 37) | aaa agg gtc agt atg ggt t | (SEQ ID NO 56) |
| caa gga tgg aat gtt ttc cct | (SEQ ID NO 38) | agg gaa aac att cca tcc | (SEQ ID NO 57) |
| | | ttg | |
| tct cta cct gaa gaa gtt | (SEQ ID NO 39) | aac ttc ttc agg tag aga | (SEQ ID NO 58) |
| ttc gaa atg gat aac gtc ctc | (SEQ ID NO 40) | gag gac gtt atc cat ttc | (SEQ ID NO 59) |
| | | gaa | |

It is understood in the art that an oligonucleotide need not be 100% complementary to its target DNA sequence to be specifically hybridizable. An oligonucleotide is specifically hybridizable when there is a sufficient degree of complimentarity to avoid non-specific binding of the oligonucleotide to non-target sequences under conditions in which specific binding is desired, i.e., under physiological conditions in the case of in vivo assays or therapeutic treatment, or in the case of in vitro assays, under conditions in which the assays are performed. The present invention encompasses oligonucleotides with sufficient specificity to effect an alteration in the condition to be treated or in the symptoms of the condition to be treated.

Antisense oligonucleotides are commonly used as research reagents, diagnostic aids, and therapeutic agents. For example, antisense oligonucleotides, which are able to inhibit gene expression with exquisite specificity, are often used by those of ordinary skill to elucidate the function of particular genes, for example to distinguish between the functions of various members of a biological pathway. This specific inhibitory effect has, therefore, been harnessed by those skilled in the art for research uses. The specificity and sensitivity of oligonucleotides is also harnessed by those of skill in the art for therapeutic uses.

A. Modified Linkages:

Specific examples of some preferred modified oligonucleotides envisioned for this invention include those containing phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioates and those with CH₂—NH—O—CH₂, CH₂—N(CH)—O—CH₂ [known as a methylene(methylimino) or MMI backbone], CH₂—O—N (CH₃)—CH₂, CH₂—N(CH₃)—N(CH₃)—CH₂ and O—N(CH₃)—CH₂—CH₂ backbones, wherein the native phosphodiester backbone is represented as O—P—O—CH₂). Also preferred are oligonucleotides having morpholino backbone structures (Summerton and Weller, U.S. Pat. No. 5,034,506). Further preferred are oligonucleotides with NR—C(*)—(H₂—CH₂, CH₂—NR—C (*)—CH₂, CH₂—CH₂—NR—C(*), C(*)—NR—CH₂—CH₂ and CH₂—C(*)—NR—CH₂ backbones, wherein “*” represents O or S (known as amide backbones; DeMesmaeker et al., WO 92/20823, published Nov. 26, 1992). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone; (U.S. Pat. No. 5,539,082).

B. Modified Nucleobases:

The oligonucleotides of the invention may additionally or alternatively include nucleobase modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g. hypoxanthine, 6-methyladenine, 5-methylcytosine, 5-hydroxymethylcytosine (HMC), glycosyl HMC and gentiobiosyl HMC, as well synthetic nucleobases, e.g., 2-aminoadenine, 2-thiouracil, 2-thiothymine, 5-bromouracil, 5-hydroxymethyluracil, 8-azaguanine, 7-deazaguanine, N⁶ (6-aminohexyl)adenine and 2,6-diaminopurine.

Depending on the purposes for which the oligomers are to be used, the RNA or DNA oligonucleotide analogs can be oligomers in which from one to all nucleotide subunits are replaced with a nucleotide analog to confer desired properties such as detectability, increased hybridization affinity, resistance to degradation by nucleases, or the ability to covalently modify a target nucleic acid. Such oligonucleotide analogs include but are not limited to oligomers comprising 2′-O-alkyl ribonucleotides, phosphorothioate or methylphosphonate internucleotide linkages, peptide nucleic acid subunits (see U.S. Pat. No. 5,714,331, in entirety), and nucleotides modified by attachment of radioactive, or fluorescent groups, groups which intercalate, cross-link or cleave a nucleic acid, or groups which alter the electronegativity or hydrophobicity of the oligomers. Nucleotide analogues which are soluble in organic solvents rather than in aqueous solution are also useful for the present invention. Methods for making and using oligonucleotides and oligonucleotide analogs such as those listed above are well known to those skilled in the art of making and using sequence-specific hybridizing oligomers.

C. Sugar Modifications:

The oligonucleotides of the invention may additionally or alternatively comprise substitutions of the sugar portion of the individual nucleotides. For example, oligonucleotides may also have sugar mimetics such as cyclobutyis in place of the pentofuranosyl group. Other preferred modified oligonucleotides may contain one or more substituted sugar moieties comprising one of the following at the 2′ position: OH, SH, SCH₃, F, OCN, OCH₃ OCH₃, OCH₃ O(CH₂)ₙ CH₃, O(CH₂)ₙ NH₂ or O(CH₂)ₙ CH₃ where n is from 1 to about 10; C.sub.1 to C.sub.10 lower alkyl, alkoxyalkoxy, substituted lower alkyl, alkaryl or aralkyl; Cl; Br; CN; CF₃ ; OCF₃; O—, S—, or N-alkyl; O—, S—, or N-alkenyl; SOCH₃; SO₂ CH₃ ; ONO₂ ; NO₂ ; N₃ ; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂ CH₂ OCH₃, also known as 2′-O-(2-methoxyethyl)] (Martin et al., Helv. Chim. Acta, 1995, 78:486). Other preferred modifications include 2′-methoxy-(2′-O—CH₃), 2′-propoxy-(2′-OCH₂ CH₂ CH₃) and 2′-fluoro-(2′-F).

D. Other Modifications:

Similar modifications may also be made at other positions on the oligonucleotide, particularly the 3′ position of the sugar on the 3′ terminal nucleotide and the 5′ position of 5′ terminal nucleotide. The 5′ and 3′ termini of an oligonucleotide may also be modified to serve as points of chemical conjugation of, e.g., lipophilic moieties (see immediately subsequent paragraph), intercalating agents Nguyen et al., U.S. Pat. No. 4,835,263,) or hydroxyalkyl groups (Helene et al., WO 96/34008, published Oct. 31, 1996).

Other positions within an oligonucleotide of the invention can be used to chemically link thereto one or more effector groups to form an oligonucleotide conjugate. An “effecter group” is a chemical moiety that is capable of carrying out a particular chemical or biological function. Examples of such effector groups include, but are not limited to, an RNA cleaving group, a reporter group, an intercalator, a group for improving the pharmacokinetic properties of an oligonucleotide, or a group for improving the pharmacodynamic properties of an oligonucleotide and other substituents having similar properties. A variety of chemical linkers may be used to conjugate an effector group to an oligonucleotide of the invention. As an example, U.S. Pat. No. 5,578,718 to Cook et al. discloses methods of attaching an alkylthio linker, which may be further derivatized to include additional groups, to ribofuranosyl positions, nucleosidic base positions, or on inter-nucleoside linkages. Additional methods of conjugating oligonucleotides to various effector groups are known in the art.

Another preferred additional or alternative modification of the oligonucleotides of the invention involves chemically linking to the oligonucleotide one or more lipophilic moieties that enhance the cellular uptake of the oligonucleotide. Such lipophilic moieties may be linked to an oligonucleotide at several different positions on the oligonucleotide. Some preferred positions include the 3′ position of the sugar of the 3′ terminal nucleotide, the 5′ position of the sugar of the 5′ terminal nucleotide, and the 2′ position of the sugar of any nucleotide. The N⁶ position of a purine nucleobase may also be utilized to link a lipophilic moiety to an oligonucleotide of the invention. Such lipophilic moieties include but are not limited to a cholesteryl moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligonucleotides comprising lipophilic moieties, and methods for preparing such oligonucleotides, are disclosed in U.S. Pat. Nos. 5,138,045; 5,218,105; and 5,459,255.

E. Chimeric Oligonucleotides:

The present invention also includes oligonucleotides which are chimeric oligonucleotides. “Chimeric” oligonucleotides or “chimeras,” in the context of this invention, are oligonucleotides which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art. By way of example, such “chimeras” may be “gapmers” i.e., oligonucleotides in which a central portion (the “gap”) of the oligonucleotide serves as a substrate for, e.g., RNase H, and the 5′ and 3′ portions (the “wings”) are modified in such a fashion so as to have greater affinity for the target RNA molecule but are unable to support nuclease activity (e.g., 2′-fluoro- or 2′-methoxyethoxy-substituted). Other chimeras include “wingmers,” that is, oligonucleotides in which the 5′ portion of the oligonucleotide serves as a substrate for, e.g., RNase H, whereas the 3′ portion is modified in such a fashion so as to have greater affinity for the target RNA molecule but is unable to support nuclease activity (e.g., 2′-fluoro- or 2′-methoxyethoxy-substituted), or vice-versa

F. Synthesis:

The oligonucleotides used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides such as the phosphorothioates and alkylated derivatives. An example of current synthetic methods are shown below, see U.S. Pat. No. 6,221,850. The oligonucleotides of the present invention were synthesized by a commercial establishment, Keystone/Biosource International, Camarillo, Calif. Oligonucleotides encompassed by the present invention can be made by any method known in the art.

1. Teachings regarding the synthesis of particular modified oligonucleotides may be found in the following U.S. patents or pending patent applications, each of which is commonly assigned with this application: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone modified oligonucleotides and the preparation thereof through reductive coupling; U.S. Pat. No. 5,457,191, drawn to modified nucleobases based on the 3-deazapurine ring system and methods of synthesis thereof, U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having .beta.-lactam backbones; U.S. Pat. No. 5,571,902, drawn to methods and materials for the synthesis of oligonucleotides; U.S. Pat. No. 5,518,718, drawn to nucleosides having alkylthio groups, wherein such groups may be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. Nos. 5,223,168, and 5,608,046, both drawn to conjugated 4′-desmethyl nucleoside analogs; U.S. Pat. Nos. 5,602,240, and 5,610,289, drawn to backbone modified oligonucleotide analogs; and U.S. Pat. No. 626,241, and U.S. Pat. No. 5,459,255, drawn to, inter alia, methods of synthesizing 2′-fluoro-oligonucleotides.

2. 5-methyl-cytosine: In 2′-methoxyethoxy-modified oligonucleotides, 5-methyl-2′-methoxyethoxy-cytosine residues are used and are prepared as follows.

(a) 2,2′-Anhydro[1-(.beta.-D-arabinofuranosyl)-5-methyluridine]: 5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) were added to DMF (300 mL). The mixture was heated to reflux, with stirring, allowing the evolved carbon dioxide gas to be released in a controlled manner. After 1 hour, the slightly darkened solution was concentrated under reduced pressure. The resulting syrup was poured into diethylether (2.5 L), with stirring. The product formed a gum. The ether was decanted and the residue was dissolved in a minimum amount of methanol (ca. 400 mL). The solution was poured into fresh ether (2.5 L) to yield a stiff gum. The ether was decanted and the gum was dried in a vacuum oven (60° C. at 1 mm Hg for 24 h) to give a solid which was crushed to a light tan powder (57 g, 85% crude yield). The material was used as is for further reactions.

(b) 2′-O-Methoxyethyl-5-methyluridine: 2,2′-Anhydro-5-methyluridine (195 g, 0.81 M), tris(2-methoxyethyl)borate (231 g, 0.98 M) and 2-methoxyethanol (1.2 L) were added to a 2 L stainless steel pressure vessel and placed in a pre-heated oil bath at 160° C. After heating for 48 hours at 155-160° C., the vessel was opened and the solution evaporated to dryness and triturated with MeOH (200 mL). The residue was suspended in hot acetone (1 L). The insoluble salts were filtered, washed with acetone (150 mL) and the filtrate evaporated. The residue (280 g) was dissolved in CH₃ CN (600 mL) and evaporated. A silica gel column (3 kg) was packed in CH₂ Cl₂/acetone/MeOH (20:5:3) containing 0.5% Et₃ NH. The residue was dissolved in CH₂ Cl₂ (250 mL) and adsorbed onto silica (150 g) prior to loading onto the column. The product was eluted with the packing solvent to give 160 g (63%) of product.

(c) 2′-O-Methoxyethyl-5′-O-dimethoxytrityl-5-methyluridine: 2′-O-Methoxyethyl-5-methyluridine (160 g, 0.506 M) was co-evaporated with pyridine (250 mL) and the dried residue dissolved in pyridine (1.3 L). A first aliquot of dimethoxytrityl chloride (94.3 g, 0.278 M) was added and the mixture stirred at room temperature for one hour. A second aliquot of dimethoxytrityl chloride (94.3 g, 0.278 M) was added and the reaction stirred for an additional one hour. Methanol (170 mL) was then added to stop the reaction. HPLC showed the presence of approximately 70% product. The solvent was evaporated and triturated with CH₃ CN (200 mL). The residue was dissolved in CHCl₃ (1.5 L) and extracted with 2×500 mL of saturated NaHCO₃ and 2×500 mL of saturated NaCl. The organic phase was dried over NaSO₄, filtered and evaporated. 275 g of residue was obtained. The residue was purified on a 3.5 kg silica gel column, packed and eluted with EtOAc/Hexane/Acetone (5:5:1) containing 0.5% Et₃ NH. The pure fractions were evaporated to give 164 g of product. Approximately 20 g additional was obtained from the impure fractions to give a total yield of 183 g (57%).

(d) 3′-O-Acetyl-2′-O-methoxyethyl-5′-O-dimethoxytrityl-5-methyluridine: 2′-O-Methoxyethyl-5′-O-dimethoxytrityl-5-methyluridine (106 g, 0.167 M), DMF/pyridine (750 mL of a 3:1 mixture prepared from 562 mL of DMF and 188 mL of pyridine) and acetic anhydride (24.38 mL, 0.258 M) were combined and stirred at room temperature for 24 hours. The reaction was monitored by thin layer chromatography (tic) by first quenching the tic sample with the addition of MeOH. Upon completion of the reaction, as judged by tic, MeOH (50 mL) was added and the mixture evaporated at 35° C. The residue was dissolved in CHCl₃ (800 mL) and extracted with 2×200 mL of saturated sodium bicarbonate and 2×200 mL of saturated NaCl. The water layers were back extracted with 200 mL of chloroform. The combined organics were dried with sodium sulfate and evaporated to give 122 g of residue (approximately 90% product). The residue was purified on a 3.5 kg silica gel column and eluted using EtOAc/Hexane (4:1). Pure product fractions were evaporated to yield 96 g (84%).

(e) 3′-O-Acetyl-2′-O-methoxyethyl-5′-O-dimethoxytrityl-5-methyl-4-triazoleuridine: A first solution was prepared by dissolving 3′-O-acetyl-2′-O-methoxyethyl-5′-O-dimethoxytrityl-5-methyluridine (96 g, 0.144 M) in CH₃ CN (700 mL) and set aside. Triethylamine (189 mL, 1.44 M) was added to a solution of triazole (90 g, 1.3 M) in CH₃ CN (1 L), cooled to −5° C. and stirred for 0.5 h using an overhead stirrer. POCl₃ was added dropwise, over a 30 minute period, to the stirred solution maintained at 0-10° C., and the resulting mixture stirred for an additional 2 hours. The first solution was added dropwise, over a 45 minute period, to the later solution. The resulting reaction mixture was stored overnight in a cold room. Salts were filtered from the reaction mixture and the solution was evaporated. The residue was dissolved in EtOAc (1 L) and the insoluble solids were removed by filtration. The filtrate was washed with 1×300 mL of NaHCO₃ and 2×300 mL of saturated NaCl, dried over sodium sulfate and evaporated. The residue was triturated with EtOAc to give the title compound. (f) 2′-O-Methoxyethyl-5′-O-dimethoxytrityl-5-methylcytidine: A solution of 3′-O-acetyl-2′-O-methoxyethyl-5′-O-dimethoxytrityl-5-methyl-4-triazoleuridine (103 g, 0.141 M) in dioxane (500 mL) and NH₄ OH (30 mL) was stirred at room temperature for 2 hours. The dioxane solution was evaporated and the residue azeotroped with MeOH (2×200 mL). The residue was dissolved in MeOH (300 mL) and transferred to a 2 liter stainless steel pressure vessel. Methanol (400 mL) saturated with NH₃ gas was added and the vessel heated to 100° C. for 2 hours (thin layer chromatography, tlc, showed complete conversion). The vessel contents were evaporated to dryness and the residue was dissolved in EtOAc (500 mL) and washed once with saturated NaCl (200 mL). The organics were dried over sodium sulfate and the solvent was evaporated to give 85 g (95%) of the title compound. (g) N⁴-Benzoyl-2′-O-methoxyethyl-5′-O-dimethoxytrityl-5-methylcytidine: 2′-O-Methoxyethyl-5′-O-dimethoxytrityl-5-methylcytidine (85 g, 0.134 M) was dissolved in DMF (800 mL) and benzoic anhydride (37.2 g, 0.165 M) was added with stirring. After stirring for 3 hours, tlc showed the reaction to be approximately 95% complete. The solvent was evaporated and the residue azeotroped with MeOH (200 mL). The residue was dissolved in CHCl₃ (700 mL) and extracted with saturated NaHCO₃ (2×300 mL) and saturated NaCl (2×300 mL), dried over MgSO₄ and evaporated to give a residue (96 g). The residue was chromatographed on a 1.5 kg silica column using EtOAc/Hexane (1:1) containing 0.5% Et₃ NH as the eluting solvent. The pure product fractions were evaporated to give 90 g (90%) of the title compound.

(h) N⁴-Benzoyl-2′-O-methoxyethyl-5′-O-dimethoxytrityl-5-methylcytidine-3′-amidite: N⁴-Benzoyl-2′-O-methoxyethyl-5′-O-dimethoxytrityl-5-methylcytidine (74 g, 0.10 M) was dissolved in CH₂ Cl₂ (1 L). Tetrazole disopropylamine (7.1 g) and 2-cyanoethoxy-tetra(isopropyl)-phosphite (40.5 mL, 0.123 M) were added with stirring, under a nitrogen atmosphere. The resulting mixture was stirred for 20 hours at room temperature (t/c showed the reaction to be 95% complete). The reaction mixture was extracted with saturated NaHCO₃ (1×300 mL) and saturated NaCl (3×300 mL) The aqueous washes were back-extracted with CH₂ Cl₂ (300 mL), and the extracts were combined, dried over MgSO₄ and concentrated. The residue obtained was chromatographed on a 1.5 kg silica column using EtOAc Hexane (3:1) as the eluting solvent. The pure fractions were combined to give 90.6 g (87%) of the title compound.

2. Bioequivalents

The compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to “prodrugs” and “pharmaceutically acceptable salts” of the oligonucleotides of the invention, pharmaceutically acceptable salts of such prodrugs, and other bioequivalents.

A. Oligonucleotide Prodrugs:

The oligonucleotides of the invention may additionally or alternatively be prepared to be delivered in a “prodrug” form. The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993.

B. Pharmaceutically Acceptable Salts:

The term “pharmaceutically acceptable salts” refers to physiologically and pharmaceutically acceptable salts of the oligonucleotides of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine. The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

3. Exemplary Utilities of the Invention

The oligonucleotides of the present invention specifically hybridize to nucleic acids (e.g., mRNAs) encoding the NK-1 receptor protein. The oligonucleotides of the present invention can be utilized as therapeutic compounds, as diagnostic tools or research reagents that can be incorporated into kits, and in purifications and cellular product preparations, as well as other methodologies, which are appreciated by persons of ordinary skill in the art.

A. Assays and Diagnostic Applications:

The oligonucleotides of the present invention can be used to detect the presence of NK-1 receptor protein-specific nucleic acids in a cell, fluid, or tissue sample. For example, radiolabeled oligonucleotides can he prepared by ³²P labeling at the 5′ end with polynucleotide kinase. (Sambrook et al., Molecular Cloning. A Laboratory Manual, Cold Spring Harbor Laboratory Press, 1989, Volume 2, pg. 10.59.) Radiolabeled oligonucleotides are then contacted with cell or tissue samples suspected of containing NK-1 receptor protein message RNA's (and thus NK-1 receptor proteins), and the samples are washed to remove unbound oligonucleotide. Radioactivity remaining in the sample indicates the presence of bound oligonucleotide, which in turn indicates the presence of nucleic acids complementary to the oligonucleotide, and can be quantified using a scintillation counter or other routine means. Expression of nucleic acids encoding these proteins is thus detected.

Radiolabeled oligonucleotides of the present invention can also be used to perform autoradiography of tissues to determine the localization, distribution and quantity of NK-1 receptor proteins for research, diagnostic or therapeutic purposes. In such studies, tissue sections are treated with radiolabeled oligonucleotide and washed as described above, then exposed to photographic emulsion according to routine autoradiography procedures. The emulsion, when developed, yields an image of silver grains over the regions expressing a NK-1 receptor protein gene. Quantitation of the silver grains permits detection of the expression of mRNA molecules encoding these proteins and permits targeting of oligonucleotides to these areas.

Analogous assays for fluorescent detection of expression of NK-1 protein nucleic acids can be developed using oligonucleotides of the present invention which are conjugated with fluorescein or other fluorescent tags instead of radiolabeling. Such conjugations are routinely accomplished during solid phase synthesis using fluorescently labeled amidites or controlled pore glass (CPG) columns. Fluorescein-labeled amidites and CPG are available from, e.g., Glen Research, Sterling Va. Other means of labeling oligonucleotides are known in the art, such as markers for detection in imaging for detection, diagnosis, delivery, or measurement.

B. Biologically Active Oligonucleotides:

The invention is drawn to the administration of oligonucleotides or other molecules having biological activity to cultured cells, isolated tissues and organs and animals. By “having biological activity,” it is meant that the oligonucleotide functions to modulate the expression of one or more genes in cultured cells isolated tissues or organs and/or animals. Such modulation can be achieved by an antisense oligonucleotide by a variety of mechanisms known in the art, including but not limited to transcriptional arrest; effects on RNA processing (capping, polyadenylation and splicing) and transportation; enhancement of cellular degradation of the target nucleic acid; and translational arrest.

In an animal other than a human, the compositions and methods of the invention can be used to study the function of one or more genes in the animal. For example, antisense oligonucleotides have been systemically administered to rats in order to study their role. In instances where complex families of related proteins are being investigated, “antisense knockdowns” (i.e., inhibition of a gene by systemic administration of antisense oligonucleotides) may represent the most accurate means for examining a specific member of the family.

The compositions and methods of the invention also have therapeutic uses in an animal, including a human, having (i.e., suffering from), or known to be or suspected of being prone to having, a disease or disorder that is treatable in whole or in part with one or more nucleic acids. The term “therapeutic uses” is intended to encompass prophylactic, palliative and curative uses wherein the oligonucleotides of the invention are contacted with animal cells either in vivo or ex vivo. When contacted with animal cells ex vivo, a therapeutic use includes incorporating such cells into an animal after treatment with one or more oligonucleotides of the invention.

For therapeutic uses, an animal suspected of having a disease or disorder which can be treated or prevented by modulating the expression or activity of the NK-1 receptor protein is, for example, treated by administering oligonucleotides in accordance with this invention. The oligonucleotides of the invention can be utilized in pharmaceutical compositions by adding an effective amount of an oligonucleotide to a suitable pharmaceutically acceptable carrier such as, e.g., a diluent. Workers in the field have identified antisense, triplex and other oligonucleotide compositions which are capable of modulating expression of genes implicated in viral, fungal and metabolic diseases. Antisense oligonucleotides have been safely administered to humans and several clinical trials are presently underway. It is thus established that oligonucleotides can be useful therapeutic instrumentalities that can be configured to be useful in treatment regimes for treatment of cells, tissues and animals, especially humans. The following U.S. patents demonstrate palliative, therapeutic and other methods utilizing antisense oligonucleotides. U.S. Pat. No. 5,135,917 provides antisense oligonucleotides that inhibit human interleukin-1 receptor expression. U.S. Pat. No. 5,098,890 is directed to antisense oligonucleotides complementary to the c-myb oncogene and antisense oligonucleotide therapies for certain cancerous conditions. U.S. Pat. No. 5,087,617 provides methods for treating cancer patients with antisense oligonucleotides. U.S. Pat. No. 5,166,195 provides oligonucleotide inhibitors of Human Immunodeficiency Virus (HIV). U.S. Pat. No. 5,004,810 provides oligomers capable of hybridizing to herpes simplex virus Vmw65 mRNA and inhibiting replication. U.S. Pat. No. 5,194,428 provides antisense oligonucleotides having antiviral activity against influenza virus. U.S. Pat. No. 4,806,463 provides antisense oligonucleotides and methods using them to inhibit HTLV-III replication. U.S. Pat. No. 5,286,717 provides oligonucleotides having a complementary base sequence to a portion of an oncogene. U.S. Pat. No. 5,276,019 and U.S. Pat. No. 5,264,423 are directed to phosphorothioate oligonucleotide analogs used to prevent replication of foreign nucleic acids in cells. U.S. Pat. No. 4,689,320 is directed to antisense oligonucleotides as antiviral agents specific to cytomegalovirus (CMV). U.S. Pat. No. 5,098,890 provides oligonucleotides complementary to at least a portion of the mRNA transcript of the human c-myb gene. U.S. Pat. No. 5,242,906 provides antisense oligonucleotides useful in the treatment of latent Epstein-Barr virus (EBV) infections.

As used herein, the term “disease or disorder” (1) includes any abnormal condition of an organism or part, especially as a consequence of infection, inherent weakness, environmental stress, that impairs normal physiological functioning; (2) excludes pregnancy per se but not autoimmune and other diseases associated with pregnancy; and (3) includes cancers and tumors. The term “known to be or suspected of being prone to having a disease or disorder” indicates that the subject animal has been determined to be, or is suspected of being, at increased risk, relative to the general population of such animals, of developing a particular disease or disorder as herein defined. For example, a subject animal “known to be or suspected of being prone to having a disease or disorder” could have a personal and/or family medical history that includes frequent occurrences of a particular disease or disorder. As another example, a subject animal “known to be or suspected of being prone to having a disease or disorder” could have had such a susceptibility determined by genetic screening according to techniques known in the art. The term “a disease or disorder that is treatable in whole or in part with one or more nucleic acids” refers to a disease or disorder, as herein defined, (1) the management, modulation or treatment thereof, and/or (2) therapeutic, curative, palliative and/or prophylactic relief therefrom, can be provided via the administration of an antisense oligonucleotide.

4. Pharmaceutical Compositions

The formulation of pharmaceutical compositions comprising the oligonucleotides of the invention, and their subsequent administration, are believed to be within the skill of those in the art.

A. Therapeutic Considerations:

In general, for therapeutic applications, a patient (i.e., an animal, including a human, having or predisposed to a disease or disorder) is administered one or more oligonucleotides, in accordance with the invention in a pharmaceutically acceptable carrier in doses ranging from 0.01 micro g to 100 g per kg of body weight depending on the age of the patient and the severity of the disorder or disease state being treated. Further, the treatment regimen may last for a period of time which will vary depending upon the nature of the particular disease or disorder, its severity and the overall condition of the patient, and may extend from once daily to once every 20 years. In the context of the invention, the term “treatment regimen” is meant to encompass therapeutic, palliative and prophylactic modalities. Following treatment, the patient is monitored for changes in his/her condition and for alleviation of the symptoms of the condition, disorder, or disease state. The dosage of the nucleic acid may either be increased in the event the patient does not respond to an acceptable degree to current dosage levels, or the dose may be decreased if an alleviation of the symptoms of the disorder or disease state is observed, or if the disorder or disease state has been ablated.

Dosing is dependent on severity and responsiveness of the disease state to be treated, with the course of treatment lasting from several days to several months, or until a cure is effected or a diminution of the disease state or symptoms is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀ s found to be effective in in vitro and in vivo animal models. In general, dosage is from 0.01 micro g to 100 g per kg of body weight, and may be given once or more daily, weekly, monthly or yearly, or even once every 2 to 20 years. An optimal dosing schedule is used to deliver a therapeutically effective amount of the oligonucleotide being administered via a particular mode of administration. Another factor to be taken into consideration is route of administration. Oral or topical dosing regimens may require higher dose levels to achieve therapeutic efficacy than systemic or intrathecal.

The present invention encompasses intrathecal administration wherein the dosage is between 0.3 and 50 nanomoles per kilogram, preferably between 15 and 30 nanomoles per kilogram most preferably between 20 and 25, and most highly preferred 22 nanomoles per kilogram. The invention also encompasses intravenous administration. Intravenous administration is a dosage between 10 and 1000 micro grams per kilogram. Preferred is between 50 and 600; highly preferred 250 and 350; most highly preferred is a dose of 300 micro grams per kilogram. For the preferred oral dosing the range of dosing is between 100 micro grams and 10 milligrams per kilogram preferred is between 50 micro grams and 5 milligrams.

The term “therapeutically effective amount,” for the purposes of the invention, refers to the amount of oligonucleotide-containing pharmaceutical composition which is effective to achieve an intended purpose without undesirable levels of side effects (such as toxicity, irritation or allergic response). Although individual needs may vary, determination of optimal ranges for effective amounts of pharmaceutical compositions is within the skill of the art. Human doses can be extrapolated from animal studies (Katocs et al., Chapter 27 In: Remington's Pharmaceutical Sciences, 18th Ed., Gennaro, ed., Mack Publishing Co., Easton, Pa., 1990). Generally, the dosage required to provide an effective amount of a pharmaceutical composition, which can be adjusted by one skilled in the art, will vary depending on the age, health, physical condition, weight, type and extent of the disease or disorder of the recipient, frequency of treatment, the nature of concurrent therapy (if any) and the nature and scope of the desired effect(s) (Nies et al., Chapter 3 In: Goodman & Gilman's The Pharmacological Basis of Therapeutics, 9th Ed., Hardman et al., eds., McGraw-Hill, New York, N.Y., 1996) Following successful treatment, it may be desirable to have the patient undergo maintenance therapy to prevent the recurrence of the disease state, wherein the nucleic acid is administered in maintenance doses, ranging from 0.01 micro g to 100 g per kg of body weight, once or more daily, to once every 20 years. For example, in the case of in individual known or suspected of being prone to an autoimmune or inflammatory condition, prophylactic effects may be achieved by administration of preventative doses, ranging from 0.01 micro g to 100 g per kg of body weight, once or more daily, to once every 20 years. In like fashion, an individual may be made less susceptible to an inflammatory condition that is expected to occur as a result of some medical treatment, e.g., graft versus host disease resulting from the transplantation of cells, tissue or an organ into the individual.

In some cases it may be more effective to treat a patient with an oligonucleotide or disrupter of the invention in conjunction with other traditional therapeutic modalities in order to increase the efficacy of a treatment regimen. In the context of the invention, the term “treatment regimen” is meant to encompass therapeutic, palliative and prophylactic modalities. For example, a patient may be treated with conventional chemotherapeutic agents, particularly those used for tumor and cancer treatment. See, generally, The Merck Manual of Diagnosis and Therapy, 15th Ed., pp. 1206-1228, Berkow et al., eds., Rahay, N.J., 1987). When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemo-therapeutic agents (e.g., 5-FU, MTX and oligonucleotide, or 5-FU, radiotherapy and oligonucleotide). Other therapeutic agents can also be used with the compounds of the present invention, for example agents that relieve pain and inflammation such as aspirin, acetaminophen, steroids, non-steroidal anti-inflammatories, capsaisin, vasoconstrictors, and vasodilators. Agents which ameliorate any condition which is treated by the invention or any condition also present in a patient can be co-administered with the present invention. Agents which reduce or prevent side effects can also be co-administered with the present invention.

B. Pharmaceutical Compositions:

Pharmaceutical compositions for the non-parenteral administration of oligonucleotides or other agents may include sterile aqueous solutions which may also contain buffers, diluents and other suitable additives. Pharmaceutically acceptable organic or inorganic carrier substances suitable for non-parenteral administration which do not deleteriously react with oligonucleotides can be used. Suitable pharmaceutically acceptable carriers include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like. The pharmaceutical compositions can be sterilized and, if desired, mixed with auxiliary agents, e.g., lubricants, preservatives, stabilizers, wetting agents, emulsifiers, salts for influencing osmotic pressure, buffers, colorings flavorings and/or aromatic substances and the like which do not deleteriously react with the oligonucleotide(s) of the pharmaceutical composition. Pharmaceutical compositions in the form of aqueous suspensions may contain substances that increase the viscosity of the suspension including, for example, sodium carboxymethylcellulose, sorbitol and/or dextran. Optionally, such suspensions may also contain stabilizers.

In one embodiment of the invention, an oligonucleotide or a disruptor is administered via the rectal mode. In particular, pharmaceutical compositions for rectal administration include foams, solutions (enemas) and suppositories. Rectal suppositories for adults are usually tapered at one or both ends and typically weigh about 2 g each, with infant rectal suppositories typically weighing about one-half as much, when the usual base, cocoa butter, is used (Block, Chapter 87 In: Remington's Pharmaceutical Sciences, 18th Ed., Gennaro, ed., Mack Publishing Co., Easton, Pa., 1990).

In a preferred embodiment of the invention, one or more oligonucleotides or disruptors are administered via oral delivery. Pharmaceutical compositions for oral administration include powders or granules, suspensions or solutions in water or non-aqueous media, capsules, sachets, troches, tablets or SECs (soft elastic capsules or “caplets”). Thickeners, flavoring agents, diluents, emulsifiers, dispersing aids, carrier substances or binders may he desirably added to such pharmaceutical compositions. The use of such pharmaceutical compositions has the effect of delivering the oligonucleotide to the alimentary canal for exposure to the mucosa thereof. Accordingly, the pharmaceutical composition can comprise material effective in protecting the oligonucleotide from pH extremes of the stomach, or in releasing the oligonucleotide over time, to optimize the delivery thereof to a particular mucosal site. Enteric coatings for acid-resistant tablets, capsules and caplets are known in the art and typically include acetate phthalate, propylene glycol and sorbitan monoleate.

Various methods for producing pharmaceutical compositions for alimentary delivery are well known in the art. See, generally, Nairn, Chapter 83; Block, Chapter 87; Rudnic et al., Chapter 89; Porter, Chapter 90; and Longer et al., Chapter 91 In: Remington's Pharmaceutical Sciences, 18th Ed., Gennaro, ed., Mack Publishing Co., Easton, Pa., 1990. The oligonucleotides of the invention can be incorporated in a known manner into customary pharmaceutical compositions, such as tablets, coated tablets, pills, granules, aerosols, syrups, emulsions, suspensions and solutions, using inert, non-toxic, pharmaceutically acceptable carriers (excipients). The therapeutically active compound should in each case be present here in a concentration of about 0.5% to about 95% by weight of the total mixture, i.e., in amounts which are sufficient to achieve the stated dosage range. The pharmaceutical compositions are prepared, for example, by diluting the active compounds with pharmaceutically acceptable carriers, if appropriate using emulsifying agents and/or dispersing agents, and, for example, in the case where water is used as the diluent, organic solvents can be used as auxiliary solvents if appropriate. Pharmaceutical compositions may be formulated in a conventional manner using additional pharmaceutically acceptable carriers as appropriate. Thus, the compositions may be prepared by conventional means with additional excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrates (e.g., starch or sodium starch glycolate); or wetting agents (e.g., sodium lauryl sulfate). Tablets may be coated by methods well known in the art. The preparations may also contain flavoring, coloring and/or sweetening agents as appropriate.

The pharmaceutical compositions, which may conveniently be presented in unit dosage form, may be prepared according to conventional techniques well known in the pharmaceutical industry. Such techniques include the step of bringing into association the active ingredient(s) with the pharmaceutically acceptable carrier(s). In general the pharmaceutical compositions are prepared by uniformly and intimately bringing into association the active ingredient(s) with liquid excipients or finely divided solid excipients or both, and then, if necessary, shaping the product.

Pharmaceutical compositions of the present invention suitable for oral administration may be presented as discrete units such as capsules, cachets or tablets each containing predetermined amounts of the active ingredients; as powders or granules; as solutions or suspensions in an aqueous liquid or a non-aqueous liquid; or as oil-in-water emulsions or water-in-oil liquid emulsions. A tablet may be made by compression or molding, optionally with one or more accessory ingredients. Compressed tablets may be prepared by compressing in a suitable machine, the active ingredients in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredients therein. Pharmaceutical compositions for parenteral, intrathecal or intraventricular administration, or colloidal dispersion systems, may include sterile aqueous solutions which may also contain buffers, diluents and other suitable additives.

C. Penetration Enhancers:

Pharmaceutical compositions comprising the oligonucleotides or disruptors of the present invention may also include penetration enhancers in order to enhance the alimentary delivery of the oligonucleotides or disruptors. Penetration enhancers may be classified as belonging to one of five broad categories, i.e., fatty acids, bile salts, chelating agents, surfactants and non-surfactants.

1. Fatty Acids: Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (a.k.a. 1-monooleoyl-rac-glycerol), dilaurin, caprylic acid, arichidonic acid, glyceryl 1-monocaprate, 1-dodecylazacycloheptan-2-one, acylcamitines, acylcholines, mono- and di-glycerides and physiologically acceptable salts thereof (i.e., oleate, laurate, caprate, myristate, palmitate, stearate, linoleate, etc.).

2. Bile Salts: The physiological roles of bile include the facilitation of dispersion and absorption of lipids and fat-soluble vitamins (Brunton, Chapter 38 In: Goodman & Gilnan's The Pharmacological Basis of Therapeutics, 9th Ed., Hardman et al., eds., McGraw-Hilt, New York, N.Y., 1996, pages 934-935). Various natural bile salts, and their synthetic derivatives, act as penetration enhancers. Thus, the term “bile salt” includes any of the naturally occurring components of bile as well as any of their synthetic derivatives.

3. Chelating Agents: Chelating agents have the added advantage of also serving as DNase inhibitors and include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines).

4. Surfactants: Surfactants include, for example, sodium lauryl sulfate, polyoxyethylene-9-lauryl ether and polyoxyethylene-20-cetyl ether.

5. Non-Surfactants: Non-surfactants include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone.

D. Carrier Compounds:

As used herein, “carrier compound” refers to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extra-circulatory reservoirs, presumably due to competition between the carrier compound and the nucleic acid for a common receptor. For example, the recovery of a partially phosphorothioated oligonucleotide in hepatic tissue is reduced when it is coadministered with polyinosinic acid, dextran sulfate, polycytidic acid or 4-acetamido-4′-isothiocyano-stilbene-2,2′-disulfonic acid.

E. Pharmaceutically Acceptable Carriers:

In contrast to a carrier compound, a “pharmaceutically acceptable carrier” (excipient) is a pharmaceutically acceptable solvent, suspending agent or any other pharmacologically inert vehicle for delivering one or more nucleic acids to an animal. The pharmaceutically acceptable carrier may be liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.). Sustained release oral delivery systems and/or enteric coatings for orally administered dosage forms are described in U.S. Pat. Nos. 4,704,295; 4,556,552; 4,309,406; and 4,309,404.

F. Miscellaneous Additional Components:

The compositions of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

G. Colloidal Dispersion Systems:

Regardless of the method by which the oligonucleotides or disruptors of the invention are introduced into a patient, colloidal dispersion systems may be used as delivery vehicles to enhance the in vivo stability of the oligonucleotides and/or to target the oligonucleotides to a particular organ, tissue or cell type. Colloidal dispersion systems include, but are not limited to, macromolecule complexes, nanocapsules, microspheres, beads and lipid-based systems including oil-in-water emulsions, micelles, mixed micelles and liposomes. A preferred colloidal dispersion system is a plurality of liposomes, artificial membrane vesicles which may be used as cellular delivery vehicles for bioactive agents in vitro and in vivo. It has been shown that large unilamellar vesicles (LUV), which range in size from 0.2-0.4 microns, can encapsulate a substantial percentage of an aqueous buffer containing large macromolecules. RNA, DNA and intact visions can be encapsulated within the aqueous interior and delivered to brain cells or other tissues in a biologically active form (Fraley et al., Trends Biochem. Sci., 1981, 6, 77). The composition of the liposome is usually a combination of lipids, particularly phospholipids, in particular, high phase transition temperature phospholipids, usually in combination with one or more steroids, particularly cholesterol. Examples of lipids useful in liposome production include phosphatidyl compounds, such as phosphatidylglycerol, phosphatidylcholine, phosphatidylserine, sphingolipids, phosphatidylethanolamine, cerebrosides and gangliosides. Particularly useful are diacyl phosphatidylglycerols, where the lipid moiety contains from 14-18 carbon atoms, particularly from 16-18 carbon atoms, and is saturated (lacking double bonds within the 14-18 carbon atom chain). Illustrative phospholipids include phosphatidylcholine, dipalmitoylphosphatidylcholine and distearoylphosphatidylcholine.

The targeting of colloidal dispersion systems, including liposomes, can be either passive or active. Passive targeting utilizes the natural tendency of liposomes to distribute to cells of the reticuloendothelial system in organs that contain sinusoidal capillaries. Active targeting, by contrast, involves modification of the liposome by coupling thereto a specific ligand such as a viral protein coat (Morishita et al., Proc. Natl. Acad. Sci. (U.S.A.), 1993, 90, 8474), monoclonal antibody (or a suitable binding portion thereof), sugar, glycolipid or protein (or a suitable oligopeptide fragment thereof), or by changing the composition and/or size of the liposome in order to achieve distribution to organs and cell types other than the naturally occurring sites of localization. The surface of the targeted colloidal dispersion system can be modified in a variety of ways. In the case of a liposomal targeted delivery system, lipid groups can be incorporated into the lipid bilayer of the liposome in order to maintain the targeting ligand in close association with the lipid bilayer. Various linking groups can be used for joining the lipid chains to the targeting ligand. The targeting ligand, which binds a specific cell surface molecule found predominantly on cells to which delivery of the oligonucleotides of the invention is desired, may be, for example, (1) a hormone, growth factor or a suitable oligopeptide fragment thereof which is bound by a specific cellular receptor predominantly expressed by cells to which delivery is desired or (2) a polyclonal or monoclonal antibody, or a suitable fragment thereof (e.g., Fab; F(ab′)₂) which specifically binds an antigenic epitope found predominantly on targeted cells. Two or more bioactive agents (e.g., an oligonucleotide and a conventional drug; two oligonucleotides) can be combined within, and delivered by, a single liposome. It is also possible to add agents to colloidal dispersion systems which enhance the intercellular stability and/or targeting of the contents thereof. Also emulsions and microemulsions are preferred that can protect an oligonucleotide through the gastrointestinal tract and increase absorption (U.S. Pat. No. 5,897,878).

5. Means of Administration

The present invention provides compositions comprising oligonucleotides or disruptors intended for administration to an animal. For purposes of the invention, unless otherwise specified, the term “animal” is meant to encompass humans as well as other mammals, as well as reptiles, amphibians, and birds. The following list is non-limiting and any means of administration can be used to treat an animal.

A. Parenteral Delivery:

The term “parenteral delivery” refers to the administration of an oligonucleotide of the invention to an animal in a manner other than through the digestive canal. Means of preparing and administering parenteral pharmaceutical compositions are known in the art (see, e.g., Avis, Chapter 84 In: Remington's Pharmaceutical Sciences, 18th Fd., Gennaro, ed., Mack Publishing Co., Easton, Pa., 1990, pages 1545-1569). Parenteral means of delivery include, but are not limited to, the following illustrative examples

1. Intravitreal injection, for the direct delivery of drug to the vitreous humor of a mammalian eye, is described in U.S. Pat. No. 5,591,720, the contents of which are hereby incorporated by reference. Means of preparing and administering ophthalmic preparations are known in the art (see, e.g., Mullins et al., Chapter 86 In: Remington's Pharmaceutical Sciences, 18th Ed., Gennaro, ed., Mack Publishing Co., Easton, Pa., 1990, pages 1581-1595).

2. Intravenous administration of antisense oligonucleotides to various non-human mammals has been described by Iversen. Systemic delivery of oligonucleotides to non-human mammals via intraperitoneal means has also been described.

3. Intraluminal drug administration, for the direct delivery of drug to an isolated portion of a tubular organ or tissue (e.g., such as an artery, vein, ureter or urethra), may be desired for the treatment of patients with diseases or conditions afflicting the lumen of such organs or tissues. To effect this mode of oligonucleotide administration, a catheter or cannula is surgically introduced by appropriate means. For example, for treatment of the left common carotid artery, a cannula is inserted thereinto via the external carotid artery. After isolation of a portion of the tubular organ or tissue for which treatment is sought, a composition comprising the oligonucleotides of the invention is infused through the cannula or catheter into the isolated segment. After incubation for from about 1 to about 120 minutes, during which the oligonucleotide is taken up by cells of the interior lumen of the vessel, the infusion cannula or catheter is removed and flow within the tubular organ or tissue is restored by removal of the ligatures which effected the isolation of a segment. Antisense oligonucleotides may also be combined with a biocompatible matrix, such as a hydrogel material, and applied directly to vascular tissue in vivo (Rosenberg et al., U.S. Pat. No. 5,593,974).

4. Intraventricular drug administration, for the direct delivery of drug to the brain of a patient, may be desired for the treatment of patients with diseases or conditions afflicting the brain. To effect this mode of oligonucleotide administration, a silicon catheter is surgically introduced into a ventricle of the brain of a human patient, and is connected to a subcutaneous infusion pump (SynchroMed® and IsoMed® infusion systems, Medtronic Inc., Minneapolis, Minn.) that has been surgically implanted in the abdominal region (Zimm et al., Cancer Research, 1984, 44, 1698. The pump is used to inject the oligonucleotides and allows precise dosage adjustments and variation in dosage schedules with the aid of an external programming device. The reservoir capacity of the pump is 18-20 mL and infusion rates may range from 0.1 mL/h to 1 mL/h. Depending on the frequency of administration, ranging from daily to monthly, and the dose of drug to he administered, ranging from 0.01 micro g to 100 g per kg of body weight, the pump reservoir may be refilled at 3-10 week intervals. Refilling of the pump is accomplished by percutaneous puncture of the self-sealing septum of the pump.

5. Intrathecal Epidural, Subdural, drug administration, for the introduction of a drug into the spinal column of a patient may be desired for the treatment of patients with diseases of the central nervous system. To effect this route of oligonucleotides administration, a silicon catheter is surgically implanted subarachnoid spinal interspace of a human patient ( for example implantation into the L3-4 lumbar cord would target the legs), and is connected to a subcutaneous infusion pump which has been surgically implanted in the upper abdominal region (Luer and Hatton, The Annals of Pharmacotherapy, 1993, 27, 912; Ettinger et al., 1978, Cancer, 41, 1270, 1978; Yaida et al., Regul. Pept., 1995, 59, 193). The pump is used to inject the oligonucleotides and allows precise dosage adjustments and variations in dose schedules with the aid of an external programming device. The reservoir capacity of the pump is 18-20 mL, and infusion rates may vary from 0.1 mL/h to 1 mL/h. Depending on the frequency of drug administration, ranging from daily to monthly, and dosage of drug to be administered, ranging from 0.01 micro g to 100 g per kg of body weight, the pump reservoir may be refilled at 3-10 week intervals. Refilling of the pump is accomplished by a single percutaneous puncture to the self-sealing septum of the pump. The distribution, stability and pharmacokinetics of oligonucleotides within the central nervous system may be followed according to known methods. Subdural administration is also envisioned other by a pump mechanism or direct injection.

To effect delivery of oligonucleotides to areas other than the brain or spinal column via this method, the silicon catheter is configured to connect the subcutaneous infusion pump to, e.g., the hepatic artery, for delivery to the liver. Infusion pumps may also be used to effect systemic delivery of oligonucleotides (Ewel et al., Cancer Research, 1992, 52, 3005; Rubenstein et al., J. Surg. Oncol., 1996, 62, 194).

6. Cutaneous, Epidermal, and Transdermal Delivery, in which pharmaceutical compositions containing drugs are applied topically, can be used to administer drugs to be absorbed by the local epidermis or dermis or for further penetration and absorption by underlying tissues, respectively. Means of preparing and administering medications topically are known in the art (see, e.g., Block, Chapter 87 In: Remington's Pharmaceutical Sciences, 18th Ed., Gennaro, ed., Mack Publishing Co., Easton, Pa., 1990, pages 1596-1609). This method is particularly preferred to treat conditions of the skin associate with NK-1 receptor activation. (See U.S. Pat. No. 5,866,168 for a discussion of skin conditions which result from substance P activation.) Such methods of delivery are known in the art, see for example U.S. Pat. No. 5,919,156 iontophoretic delivery; U.S. Pat. No. 5,582,598 delivery pen; U.S. Pat. No. 5,851,549 transdermal patch. Similar methods can be used for delivery to the ear, using drops or similar oil or viscous solution.

7. Vaginal Delivery provides local treatment and avoids first pass metabolism, degradation by digestive enzymes, and potential systemic side-effects. This mode of administration may be preferred for antisense oligonucleotides targeted to pathogenic organisms for which the vagina is the usual habitat, e.g., Trichomonas vaginalis. In another embodiment, antisense oligonucleotides to genes encoding sperm-specific antibodies can be delivered by this mode of administration in order to increase the probability of conception and subsequent pregnancy. Vaginal suppositories (Block, Chapter 87 In: Remington's Pharmaceutical Sciences, 18th Ed., Gennaro, ed., Mack Publishing Co., Easton, Pa., 1990, pages 1609-1614) or topical ointments can be used to effect this mode of delivery.

8. Intravesical Delivery provides local treatment and avoids first pass metabolism, degradation by digestive enzymes, and potential systemic side-effects. However, the method requires urethral catheterization of the patient and a skilled staff. Nevertheless, this mode of administration may be preferred for antisense oligonucleotides targeted to pathogenic organisms, such as T. vaginalis, which may invade the urogenital tract.

9. Inhalation nasal delivery provides both topical delivery to the nose, or throat, bronchi, and lungs, and systemic delivery via absorption through the nose or lungs to the circulatory system. A nasal delivery system can include nasal sprays and inhalation devices can include nebulizers or other forms of inhalation devices. See, for example, U.S. Pat. Nos. 6,267,155 6,257,233; 6,138,668; and 5,994,314.

10. Intra-articular delivery: Injection can be made in the bursa of a joint or intra-articular. This method may be preferred in arthritis or other condition of the bones or joints. The dosage for intra articular delivery is preferably in the range of 5 to 500 nanomoles.

11. Cutaneous Delivery, the oligonucleotides of the present can be delivered to the skin or through the skin by means of a cream or salve formulated by techniques known to one of skill in the art and incorporating carrier and absorption enhances such as are described above. Also useful in the present invention are methods of transdermal delivery such as patches, iontophoretic devices, and microneedle arrays.

B. Alimentary Delivery:

The term “alimentary delivery” refers to the administration, directly or otherwise, to a portion of the alimentary canal of an animal. The term “alimentary canal” refers to the tubular passage in an animal that functions in the digestion and absorption of food and the elimination of food residue, which runs from the mouth to the anus, and any and all of its portions or segments, e.g., the oral cavity, the esophagus, the stomach, the small and large intestines and the colon, as well as compound portions thereof such as, e.g., the gastrointestinal tract. Thus, the term “alimentary delivery” encompasses several routes of administration including, but not limited to, oral, rectal, endoscopic and sublingual/buccal administration. A common requirement for these modes of administration is absorption over some portion or all of the alimentary tract and a need for efficient mucosal penetration of the nucleic acid(s) so administered.

1. Buccal/Sublingual Administration

Delivery of a drug via the oral mucosa has several desirable features, including, in many instances, a more rapid rise in plasma concentration of the drug than via oral delivery (Harvey, Chapter 35 In: Remington's Pharmaceutical Sciences, 18th Ed., Gennaro, ed., Mack Publishing Co., Easton, Pa., 1990, page 711). Furthermore, because venous drainage from the mouth is to the superior vena cava, this route also bypasses rapid first-pass metabolism by the liver. Both of these features contribute to the sublingual route being the mode of choice for nitroglycerin (Benet et al., Chapter 1 In: Goodman & Gilman's The Pharmacological Basis of Therapeutics, 9th Ed., Hardman et al., eds., McGraw-Hill, New York, N.Y., 1996, page 1).

2. Endoscopic Administration:

Endoscopy can be used for drug delivery directly to an interior portion of the alimentary tract. For example, endoscopic retrograde cystopancreatography (ERCP) takes advantage of extended gastroscopy and permits selective access to the biliary tract and the pancreatic duct (Hirahata et al., Gan To Kagaku Ryoho, 1992, 19(10 Suppl.):1591). However, the procedure is unpleasant for the patient, and requires a highly skilled staff.

3. Rectal Administration:

Drugs administered by the oral route can often be alternatively administered by the lower enteral route, i.e., through the anal portal into the rectum or lower intestine. Rectal suppositories, retention enemas or rectal catheters can be used for this purpose and may be preferred when patient compliance might otherwise be difficult to achieve (e.g., in pediatric and geriatric applications, or when the patient is vomiting or unconscious). Rectal administration may result in more prompt and higher blood levels than the oral route, but the converse may be true as well (Harvey, Chapter 35 In: Remington's Pharmaceutical Sciences, 18th Ed., Gennaro, ed., Mack Publishing Co., Easton, Pa., 1990, page 711). Because about 50% of the drug that is absorbed from the rectum will bypass the liver, administration by this route significantly reduces the potential for first-pass metabolism (Benet et al., Chapter 1 In: Goodman & Gilman's The Pharmacological Basis of Therapeutics, 9th Ed., Hardman et al., eds., McGraw-Hill, New York, N.Y., 1996).

4. Oral Administration

The preferred method of administration is oral delivery, which is typically the most convenient route for access to the systemic circulation. Absorption from the alimentary canal is governed by factors that are generally applicable, e.g., surface area for absorption, blood flow to the site of absorption, the physical state of the drug and its concentration at the site of absorption (Benet et al., Chapter 1 In: Goodman & Gilman's The Pharmacological Basis of Therapeutics, 9th Ed., Hardman et al., eds., McGraw-Hill, New York, N.Y., 1996, pages 5-7). A significant factor which may limit the oral bioavailability of a drug is the degree of “first pass effects.” For example, some substances have such a rapid hepatic uptake that only a fraction of the material absorbed enters the peripheral blood. The compositions and methods of the invention circumvent, at least partially, such first pass effects by providing improved uptake of nucleic acids and thereby, e.g., causing the hepatic uptake system to become saturated and allowing a significant portion of the nucleic acid so administered to reach the peripheral circulation. Additionally or alternatively, the hepatic uptake system is saturated with one or more inactive carrier compounds prior to administration of the active nucleic acid.

For the purpose of oral administration, oligonucleotides can be administered using any of the protection mechanisms stabilizers, carriers, or penetration enhancers described above, alone or in admixture. Oral dosage is between 0.5 and 5 mg per kg, depending on the condition to be treated and the severity of the condition. Preferably between 0.75 and 4mg per kg, most preferably between 1 and 3 mg/kg Dosage can be adjusted by monitoring symptoms and also by checking normal responses to painful or inflammatory stimuli at a location distant from that affected by the condition to be treated.

The following examples illustrate the invention and are not intended to limit the same. Those skilled in the art will recognize, or be able to ascertain through routine experimentation, numerous equivalents to the specific substances and procedures described herein. Such equivalents are considered to be within the scope of the present invention. The references cited in the application are herein incorporated by reference in their entirety.

### EXAMPLES

The following examples describe experiments documenting the utility of the oligonucleotides of the invention for the treatment of pathological conditions. The animal models used are well known in the art and accepted to predictive of human and animal diseases and disorders. The models and their uses are discribed in the following articles published in peer reviewed journals.

Mosconi T. Kruger L. Fixed-diameter polyethylene cuffs applied to the rat sciatic nerve induce a painful neuropathy: ultrastructural morphometric analysis of axonal alterations. Pain. 64(l):37-57, 1996

Butler S H. Godefroy F. Besson J M. Weil-Fugazza J. A limited arthritic model for chronic pain studies in the rat. Pain. 48(1):73-81, 1992

Laneuville O. Corey E J. Couture R. Pace-Asciak C R. Hepoxilin A3 increases vascular permeability in the rat skin. Eicosanoids. 4(2):95-7, 1991

Dubuisson D. Dennis S G. The formalin test: a quantitative study of the analgesic effects of morphine, meperidine, and brain stem stimulation in rats and cats. Pain. 4(2):161-74, 1977

Hayes A G. Skingle M. Tyers M B. Effects of single doses of capsaicin on nociceptive thresholds in the rodent. Neuropharmacology. 20(5):505-11, 1981

Coutinho S V. Meller S T. Gebhart G F. Intracolonic zymosan produces visceral hyperalgesia in the rat that is mediated by spinal NMDA and non-NMDA receptors. Brain Research. 736(1-2):7-15, 1996

In the following experiments the oligonucleotide with the sequence 5′gac gtt atc cat ttt ggg g3′ (SEQ ID NO 11) was used. When the oligonucleotide was administered to the central nervous system the backbone was the natural sugar with no substitutions. When administered systemically, a phosphate was added to the backbone to reduce degradation. Methods of synthesis are described above.

### Example 1

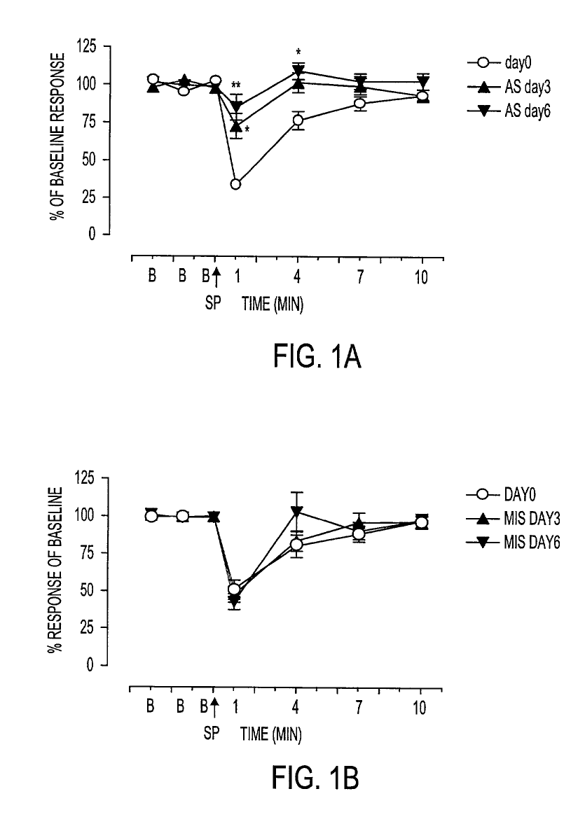

The effect of intrathecal (spinal) administration of NK-1 receptor antisense oligonucleotide on substance P-induced thermal hyperalgesia is shown in FIG. 1.

Latency to thermal stimulation (baseline (B) recordings) was assessed prior to intrathecal injection of substance P (SP, indicated by the arrow in both upper and lower panels). On day 0 (before oligonucleotide treatment has begun), SP (6.7 nmol/10 microlitres) produced a decrease in thermal latency to response indicating the occurrence of hyperalgesia (exaggerated response to noxious or painful stimulation, hollow symbols on both upper and lower panels). Oligonucleotide treatment (75 micro grams/10 microlitres, Q12h for 6 days) or ASCF control (artificial cerebral spinal fluid) was commenced subsequent to assessment of substance P-induced thermal hyperalgesia on day 0. Intrathecal SP (6.7 nmol/10 microlitres, Q12h for 6 days) was administered, in addition to the oligonucleotide, to stimulate NK-1 receptor turnover. Three days after twice-daily injections of antisense (AS) were begun, SP-induced thermal hyperalgesia was attenuated (upper graph, triangles). Six days after beginning AS treatment, there was a further decrease in the response to SP (upper graph inverted triangles). The lower panel shows that missense (MIS) had no effect on the SP-induced thermal hyeralgesia at either 3 or 6 days after oligonucleotide administration. Statistical analysis was performed by a two way ANOVA with time and treatment as independent measures. Time: F(6,21)=10.234, p<0.01, Treatment: F(6,21)=8.245, p<0.05. Post hoc analysis with Tukey's revealed significant difference compared to control values at discrete time points, *p<0.05.

These data show that the effect of AS was not mimicked by MIS, and that the ligand for the receptor has diminished effectiveness, consistent with loss of NK-1 receptors.

### Example 2

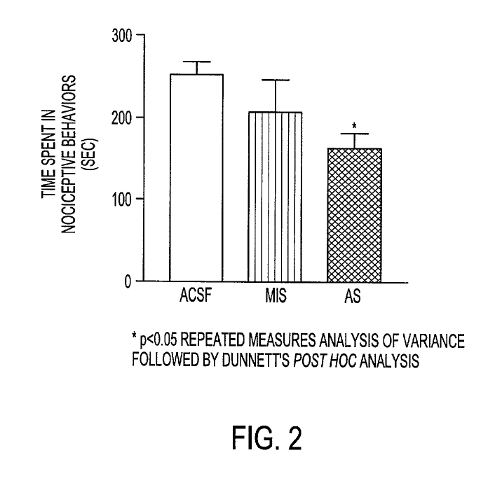

The effect of intrathecal administration of NK-1 receptor antisense oligonucleotide on the nociceptive responses induced by capsaicin is shown in FIG. 2. This test consists of injecting a dilute solution of the active ingredient of hot peppers, capsaicin subcutaneously in the plantar surface of the rat hind paw. Capsaicin, C₁₈H₂₇NO₃, is a known painful stimulus, especially of c-fibers, which are associated with substance P.

Capsaicin produces nociceptive (pain) behaviors over a period of about 5 minutes. The index of nociceptive behavior was assessed based a weighted pain intensity scale, where the animal is given a value of 0 for showing no signs of favoring of the injected paw, 1—favoring of hind paw such that it is resting lightly on the floor, 2—elevation of hind paw, 3—licking or vigorously shaking the hind paw. The numerical ratings were calculated by multiplying the amount of time the rats spent in each category by the weighted factor indicated above. This was expressed by the following formula: (time spent in category 0*0+time spent in category 1*1+time spent in category 2*2+time spent in category 3*3)/180 seconds. Oligonucleotides or ASCF control (artificial cerebral spinal fluid) were administered for 7 days prior to testing by continuous infusion (2 micro grams/microlitres/h) via Alzet™ minipumps attached to intrathecal catheters for spinal delivery. Rats also underwent constriction of the sciatic nerve on the contralateral side 7 days prior to the capsaicin test to stimulate NK-1 receptor turnover. The figure shows that there is less time spent in nociceptive behavior in animals treated for several days with AS compared to controls (treated with artificial cerebrospinal fluid, ACSF) or animals receiving given missense (MIS) oligonucleotides. Statistical analysis was performed by a repeated measures ANOVA, F(2,17)=9.452, p<0.05. Post hoc analysis with Dunnett's revealed significant difference compared to control values, *p<0.05.

These data suggest that NK-1 receptor antisense treatment may be effective in treating tonic pains. Some of these pains may also be considered as “nociceptive” pains by clinicians.

### Example 3

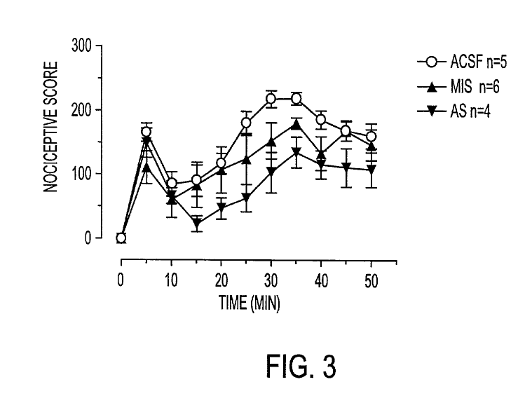

The effect of intrathecal administration of NK-1 receptor antisense oligonucleotide on the nociceptive responses induced by formalin is shown in FIG. 3.

The formalin test is similar in some ways to the capsaicin test, in that a noxious chemical is injected under the skin. In the case of formalin, however, this produces nociceptive behavior lasting up to one hour, and thus represents a longer-term tonic pain. The pain score was determined using the same parameters outlined above for the capsaicin test. Oligonucleotides or ASCF control (artificial cerebral spinal fluid) were administered for 7 days prior to testing by continuous infusion (1 micro grams/microlitres/h) via Alzet® minipumps attached to intrathecal catheters for spinal delivery. The figure shows that the nociceptive scores are less in rats which had been receiving NK-1 receptor antisense (AS, inverted triangles) intrathecally for several days. Missense-treated rats (MIS, triangles) did not show this decrease. Statistical analysis was performed by a two way ANOVA with time and treatment as independent measures. Time: F(9,44)=8.264, p<0.05, Treatment: F(9,44)=7.254, p<0.05. Post hoc analysis with Tukey's revealed significant difference compared to control values at discrete time points, *p<0.05. FIG. 3B shows that a lower dose of AS was without effect. As above, these data suggest that antisense treatment may be effective in treating tonic pains.

These data suggest that NK-1 receptor antisense treatment may be effective in treating tonic, more persistent pains and chronic pain.

### Example 4

The effect of intrathecal administration of NK-1 receptor antisense oligonucleotide on the nociceptive responses induced by formalin.

Antisense (AS) oligonucleotide or ASCF control (artificial cerebral spinal fluid) was administered for 7 days prior to testing by continuous infusion (0.45 micro grams/microlitres/h) via Alzet® minipumps attached to intrathecal catheters for spinal delivery. The figure shows that there is no attenuation of formalin-induced nociceptive (painful) responses when the concentration of AS is lower. Statistical analysis was performed by a two way ANOVA with time and treatment as independent measures. Time: F(9,17)=7.985, p<0.05, Treatment: F(9,17)=2.541, p>0.05.

These data suggest that effectiveness of NK-1 receptor antisense treatment is dose-dependent.

### Example 5

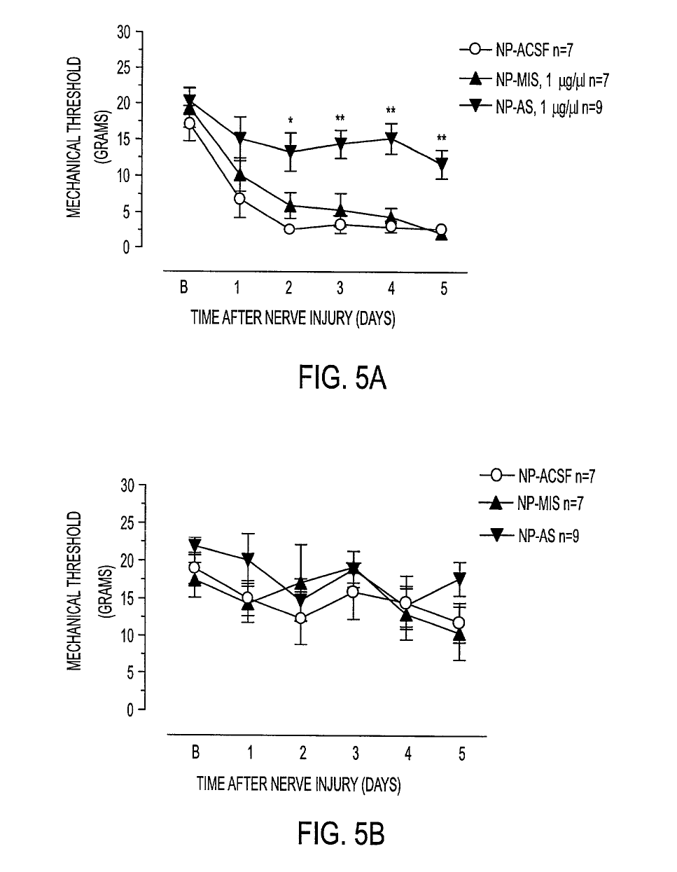

The effect of intrathecal administration of NK-1 receptor antisense oligonucleotide in preventing mechanical allodynia a model of neuropathic pain is shown in FIG. 5.

Neuropathic pain is a continuous, permanent pain that results from nerve damage. One common feature of neuropathic pain is mechanical allodyina (a painful response to a non-painful stimulus). Humans with neuropathic pain cannot wear clothing on affected regions of the skin or cannot stand in air currents, because their sensitivity is so profound. Neuropathic pain was induced by constriction of the sciatic nerve. Mechanical response thresholds (grams of force) were assessed by application of von Frey hairs to the plantar surface of the ipsilateral hind paw for all groups prior to sciatic nerve constriction surgery (baseline values: B). Oligonucleotides or ASCF control (artificial cerebral spinal fluid) were administered for 2 days prior to surgery, and throughout the time course of testing, by continuous infusion (1 micro grams/microlitres/h) via Alzet® minipumps attached to intrathecal catheters for spinal delivery. The upper panel shows that in control animals (ACSF) the mechanical response thresholds were lower than baseline at all time points following sciatic nerve constriction indicating the induction of mechanical allodynia. Rats that had been receiving antisense (AS) did not show a decrease in their threshold of mechanical stimulation, however, rats treated with missense (MIS) were not different than controls. Statistical analyses on mechanical thresholds were performed using the non-parametric Friedman's repeated measures analysis of variance on ranks followed by Wilcoxon signed rank test for post hoc comparisons. Results indicate a significant main effect of time for the vehicle-treated (χ²(⁷)=24.1, p<0.01) and MIS-treated (χ²(⁷)=7.54, p<0.05) groups. The lower panel illustrates the mechanical response thresholds are not affected in the contralateral hind paw.

This figure shows that the development of neuropathic pain is prevented in rats receiving AS.

### Example-6

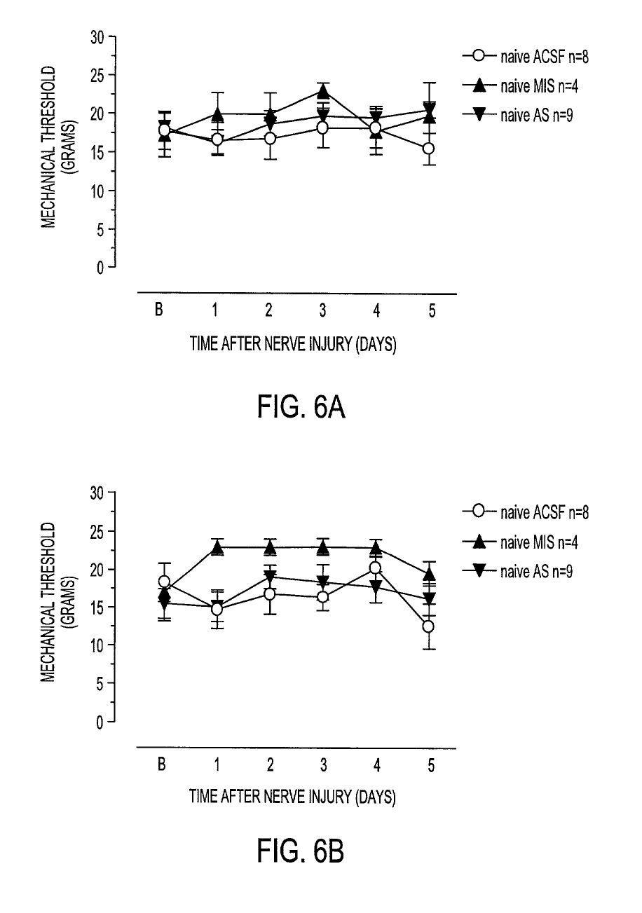

The effect of intrathecal administration of NK-1 receptor antisense oligonucleotide on mechanical response thresholds in naive rats is shown in FIG. 6.

Oligonucleotides or ASCF control (artificial cerebral spinal fluid) were administered for 2 days prior to surgery, and throughout the time course of testing, by continuous infusion (1 micro grams/microlitres/h) via Alzet® minipumps attached to intrathecal catheters for spinal delivery. The figure shows that in animals receiving no surgery, in which allodynia does not develop, prolonged administration of ACSF, antisense (AS) and missense (MIS) had no effect on withdrawal threshold of either hind limb.

This suggests that AS treatment has no effect on sensory perception under normal circumstances.

### Example-7

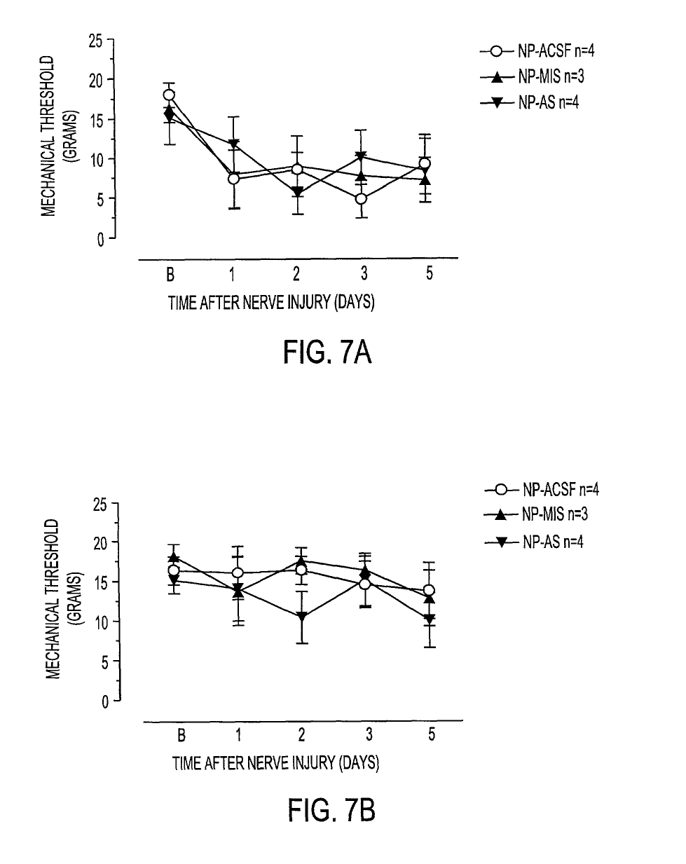

Effect of intrathecal delivery of NK-1 receptor antisense oligonucleotide to a different spinal segmental level in preventing mechanical allodynia in a model of neuropathic pain is shown in FIG. 7.

Neuropathic pain was induced by constriction of the sciatic nerve. Mechanical response thresholds (grams of force) were assessed by application of von Frey hairs to the plantar surface of the ipsilateral hind paw for all groups prior to sciatic nerve constriction surgery (baseline values: B). Oligonucleotides or ASCF control (artificial cerebral spinal fluid) were administered for 2 days prior to surgery, and throughout the time course of testing, by continuous infusion (1 micro grams/microlitres/h) via Alzet® minipumps attached to intrathecal catheters for spinal delivery. The upper panel shows that in control animals (ACSF) the mechanical response thresholds were lower than baseline at all time points following sciatic nerve constriction indicating the induction of mechanical allodynia. It also shows that when intrathecal administration was to the sacral rather than to the lumbar spinal cord, ie. the delivery catheter was just less than 1 cm from the level of the spinal cord where limb reflexes are mediated, AS treatment failed to prevent the development of allodynia.

These results show that administration of AS to a neighboring spinal level does not spread far from the site of delivery. This is important as it indicates that intrathecal AS is unlikely to spread to other sites, where it could conceivably produce side effects.

### Example 8

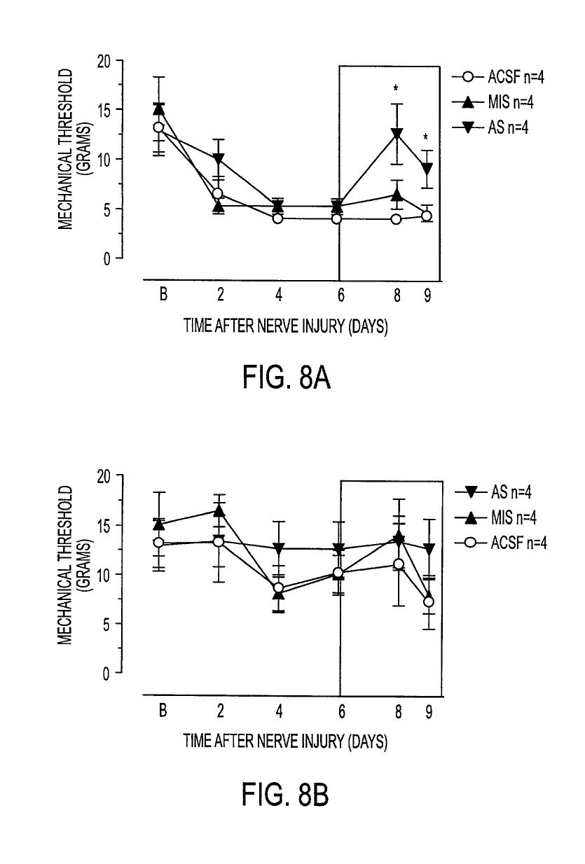

Effect of intrathecal delivery of NK-1 receptor antisense oligonucleotide in alleviating established mechanical allodynia in a model of neuropathic pain is shown in FIG. 8.

Neuropathic pain was induced by constriction of the sciatic nerve. Mechanical response thresholds (grams of force) were assessed by application of von Frey hairs to the plantar surface of the ipsilateral hind paw for all groups prior to sciatic nerve constriction surgery (baseline values: B). Oligonucleotides or ASCF control (artificial cerebral spinal fluid) was administered for 72 h by continuous infusion (2 micro grams/microlitres/h) via Alzet® minipumps attached to intrathecal catheters for spinal delivery starting 6 days following nerve constriction (once the mechanical allodynia was established in all groups). The upper panel shows that in control animals (ACSF) the mechanical response thresholds were lower than baseline at all time points following sciatic nerve constriction indicating the induction of mechanical allodynia. Intrathecal administration of antisense (AS) reversed allodynia in the animal model of neuropathic pain whereas missense (MIS) had no effect. The lower panel shows that the mechanical response thresholds were not effected by either the surgery or treatment on the contralateral side.

The data suggest that even after allodynia has developed, AS treatment can reverse the chronic pain, the administration has no effect on the contralateral side, and is not mimicked by MIS or by intrathecal administration per se.

### Example 9

The effect of intrathecal delivery of a lower dose of NK-1 receptor antisense oligonucleotide in alleviating established mechanical allodynia in a model of neuropathic pain is shown in FIG. 7.

Neuropathic pain was induced by constriction of the sciatic nerve. Mechanical response thresholds (grams of force) were assessed by application of von Frey hairs to the plantar surface of the ipsilateral hind paw for all groups prior to sciatic nerve constriction surgery (baseline values: B). Oligonucleotides or ASCF control (artificial cerebral spinal fluid) was administered for 6 days by continuous infusion (0.45 micro grams/microlitres/h) via Alzet® minipumps attached to intrathecal catheters for spinal delivery starting 7 days following nerve constriction (once the mechanical allodynia was established in all groups). The figure shows that the lower dose of AS had no effect in reversing the decrease in mechanical thresholds.

Thus, the anti-allodynic effect of AS seen in the previous example is related to the dose given.

### Example 10

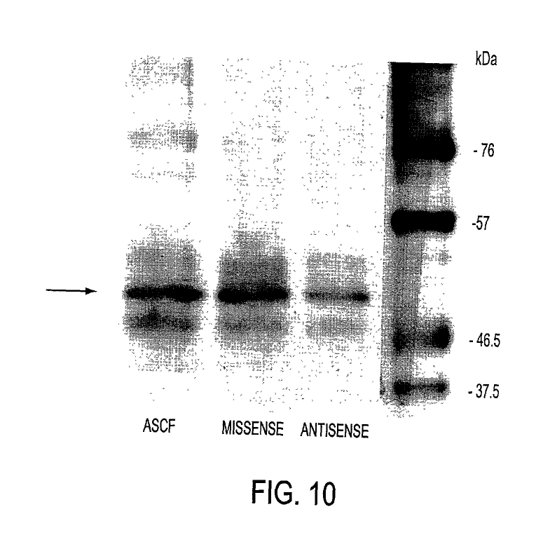

The effect of intrathecal delivery of NK-1 receptor antisense oligonucleotide on protein expression in a model of neuropathic pain is shown in FIG. 10.

Oligonucleotides or ASCF control (artificial cerebral spinal fluid) was administered for 48 h prior to sciatic nerve constriction and continuously for 6 days (2 micro grams/microlitres/h) via Alzet® minipumps attached to intrathecal catheters for spinal delivery. Rats were sacrificed by decapitation and lumbar spinal cords were dissected and processed for Western blotting analysis. Samples were resolved using 10% Tris-glycine gels and the proteins were electroblotted onto nitrocellulose membranes. Molecular mass markers are presented in the right column. The arrow indicates the NK-1 receptor. The figure demonstrates that antisense (AS), but not missense (MIS) treatment significantly attenuates the protein expression compared to ACSF control values.

These data demonstrate that the AS sequence significantly attenuates the translation of mRNA for the NK-1 receptor into protein.

### Example 11

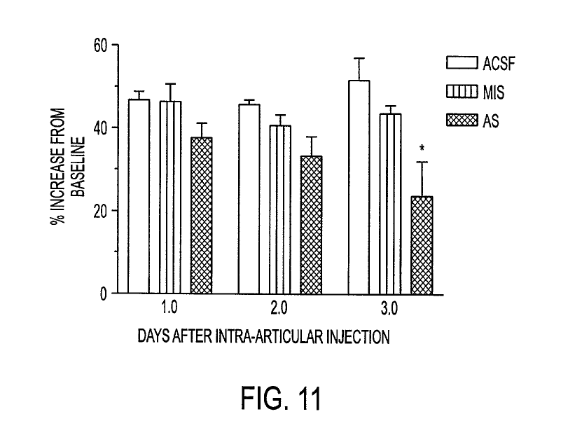

The effect of intrathecal delivery of NK-1 receptor antisense oligonucleotide in a model of arthritis is shown in FIG. 11.

Complete Freund's Adjuvant (CFA) injection into the ankle joint of the rat produces two responses, an inflammatory response that begins within 24 hours and lasts approximately one week, and a later inflammatory response combined with symptoms modelling arthritis. Oligonucleotides or ASCF control (artificial cerebral spinal fluid) was administered for 48 h prior to CFA injection and continuously for the testing days (1 micro grams/microlitres/h) via Alzet® minipumps attached to intrathecal catheters for spinal delivery. This figure shows that the magnitude of inflammation, measured as ankle circumference, is less in animals treated with antisense (AS) than in those treated with ACSF or missense (MIS). Statistical analysis was performed using a one way ANOVA and post hoc analysis using the Dunnett's test.

These data suggest that AS treatment may be effective clinically for the treatment of inflammatory disorders, such as those listed above.

### Example 12

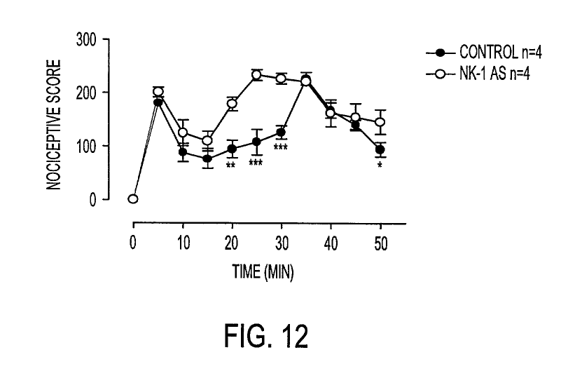

The effect of systemic administration of NK-1 receptor antisense oligonucleotide on the nociceptive responses induced by formalin is shown FIG. 12.

The formalin test produces a biphasic nociceptive behavior that lasts up to one hour that is believed to represent a tonic, persistent pain. The index of nociceptive behavior is assessed based a weighted pain intensity scale, where the animal is given a value of 0 for showing no signs of favoring of the injected paw, 1—favoring of hind paw such that it is resting lightly on the floor, 2—elevation of hind paw, 3—licking or vigorously shaking the hind paw. The numerical ratings were calculated by multiplying the amount of time the rats spent in each category by the weighted factor indicated above for each 5 minute interval over a one hour time period. This was expressed by the following formula: (time spent in category 0*0+time spent in category 1*1+time spent in category 2*2+time spent in category 3*3)/300 seconds. Antisense (AS) oligonucleotide (300 micro grams/kg, Q12 h, intraperitoneal) or saline was administered for 6 days prior to testing. The figure shows that the nociceptive scores are less in rats which had been receiving NK-1 receptor AS (solid symbols) for several days. Statistical analysis was performed by a two way ANOVA with time and treatment as independent measures. Time: F=9.966, p<0.001, Treatment: F=31.85, p<0.001. Post hoc analysis with Tukey's revealed significant difference compared to control values at discrete time points, *p<0.05, **p<0.01, ***p<0.001.

These data suggest that peripheral administration of NK-1 receptor antisense treatment may be effective in treating tonic and persistent pains.

### Example 13

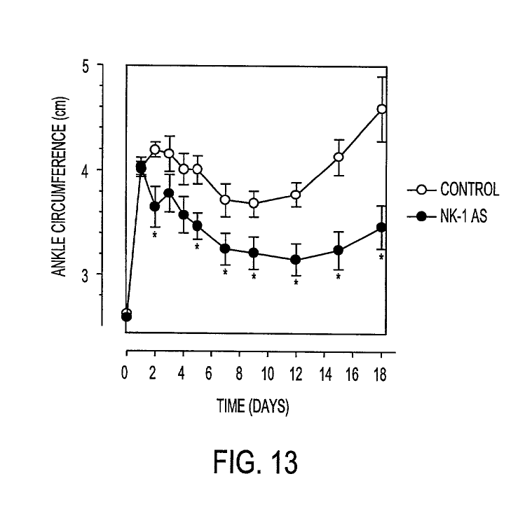

The effect of systemic administration of NK-1 receptor antisense oligonucleotide in a model of arthritis is shown in FIG. 13. Complete Freund's Adjuvant (CFA) injection (13.6 micro grams/25 microlitres) into the ankle joint of the rat produces two responses, an inflammatory response that begins within 24 hours and lasts approximately one week, and a later inflammatory response combined with symptoms modeling arthritis. Antisense (AS) oligonucleotide (300 micro grams/kg, Q12 h, intraperitoneal) or saline was administered for 2 days prior to CFA injection and throughout the time course of testing. In control animals CFA induces two phases of response as assessed by ankle circumference; the first is maximal at 48 h after injection and the second at day 18. This figure shows that the magnitude of inflammation, measured as ankle circumference, in the first phase as well as the second phase is less in animals treated with AS compared than in those treated with ACSF or missense (MIS). Statistical analysis was performed using a two way ANOVA and post hoc analysis using the tukey's test.

These data suggest that peripheral AS treatment may be effective clinically for the treatment of arthritic disorders.

### Example 14

The effect of systemic administration of NK-1 receptor antisense oligonucleotide in a model of arthritis.

Complete Freund's Adjuvant (CFA) was injected into the ankle joint of the rat and the flexibility of the affected joint was assessed on day 18 during the arthritic phase of the model. This was accomplished by measuring the difference between the extension and flexion of the affected joint. Antisense (AS) oligonucleotide (300 micro grams/kg, Q12 h, intraperitoneal) or saline was administered for 2 days prior to CFA injection and for the subsequent 18 days. The figure shows that in saline-treated animals the CFA injection significantly attenuated joint flexibility compared to the measurements taken prior to CFA administration; however, AS treated animals exhibit significantly improved flexibility compared to saline controls and flexibility was not significantly different compared to naïve animals. Statistical analysis was performed using a one way ANOVA and post hoc analysis using the Dunnett's test.

These data suggest that peripheral AS treatment may be effective clinically for the treatment of arthritic disorders.

### Example 15

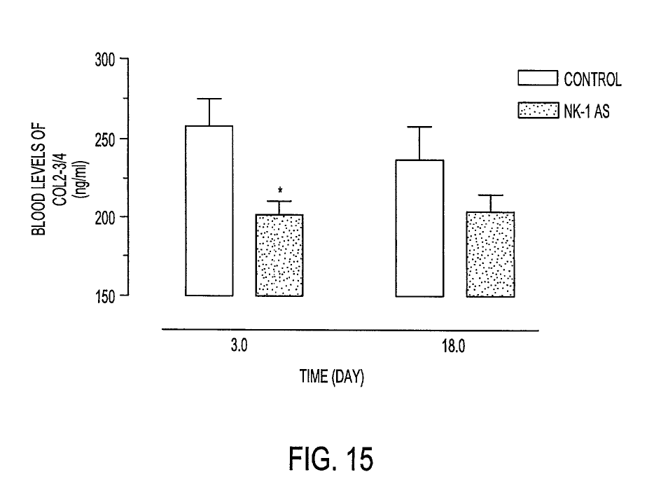

The effect of systemic administration of NK-1 receptor antisense oligonucleotide in a model of arthritis is shown in FIG. 15. Complete Freund's Adjuvant (CFA) was injected into the ankle joint of the rat and the levels of collagen breakdown products were assessed on days 3 and 18 following CFA injection. This was accomplished by measuring collagen degradation in the plasma of blood samples taken from the tail vein of rats while lightly anesthetized with halothane anesthesia. Antisense (AS) oligonucleotide (300 micro grams/kg, Q12 h, intraperitoneal) or saline was administered for 2 days prior to CFA injection and for the subsequent 18 days. The figure shows that there is a significant attenuation of the collagen breakdown products in antisense treated rats compared to saline controls. (Pervious experiments have identified that the levels of collagen breakdown products are maximal at day 3-5 after CFA injection). Statistical analysis was performed using an unpaired t-test.

These data suggest that peripheral AS treatment may be effective clinically for the treatment of arthritic disorders.

### Example 16

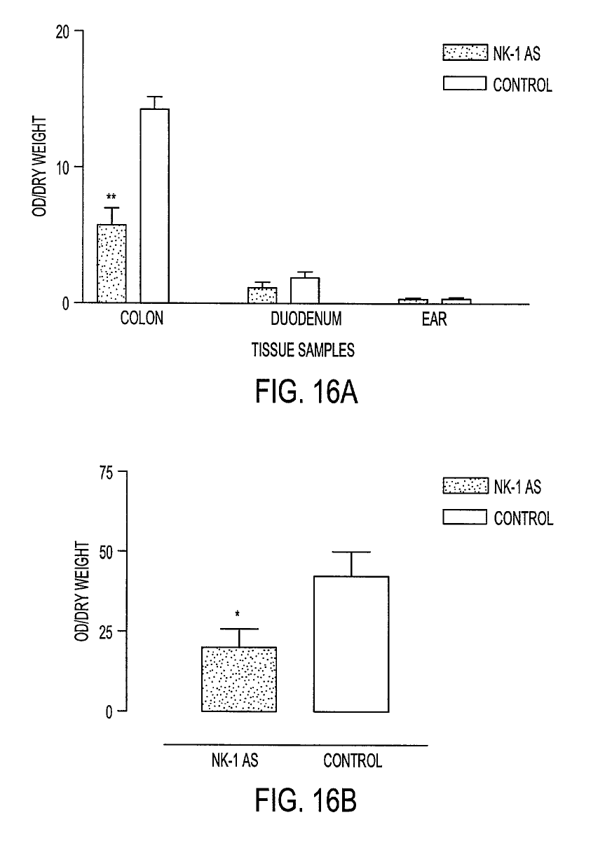

The effect of systemic administration of NK-1 receptor antisense in a model of inflammatory bowel disease is shown in FIG. 16. Inflammatory bowel disease (IBD) is a generic term used to describe major clinical entities including Crohn's disease and ulcerative colitis. IBD was simulated by rectal administration of zymosan, a complement-activating substance. Antisense (AS) oligonucleotide (300 micro grams/kg, Q12 h, intraperitoneal) or saline was administered for 6 days prior to zymosan administration. Three hours after administration of zymosan, rats are injected with Evans blue dye which enables the assessment of plasma extravasation of the colon (degree of inflammation). The figure shows that zymosan-induced plasma extravasation was significantly lower in AS-treated compared to control animals. Statistical analysis was performed using an unpaired t-test.

These data suggest that systemic administration of oligonucleotides directed against the NK-1 receptor has therapeutic potential for attenuating the severity of diseases defined under the category of IBD, such as Crohn's Disease, and ulcerative colitis.

### Example 17

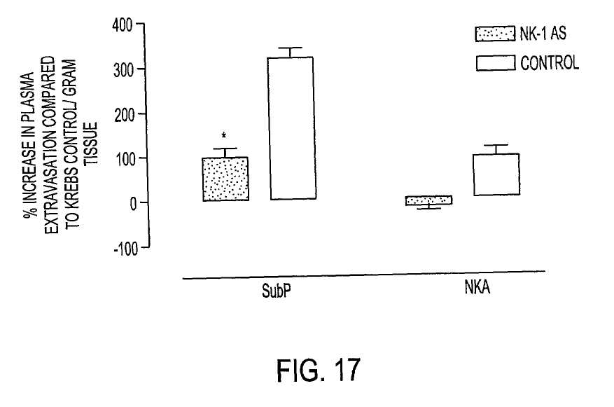

The effect of systemic administration of NK-1 receptor antisense oligonulceotide in decreasing the inflammatory response following intradermal injections of neuropeptides is shown in FIG. 17.

Intradermal injection of substance P and neurokinin A produce a wheal and flare inflammatory reaction. Antisense (AS) oligonucleotide (300 micro grams/kg, Q12 h, intraperitoneal) or saline was administered for 6 days prior to experimentation. Substance P or neurokinin A was injected into the dermis of the skin (6.7 nmol/100 microlitres) after intravenous administration of evans blue dye which allows for assessment of neuropeptide-induced plasma extravasation. The figure shows that substance P-induced plasma extravasation is significantly reduced in AS-treated rats compared to control values. Statistical analysis was performed using an unpaired t-test.

These data suggest that AS oligonucleotide reduction of the NK-1 receptor may be beneficial for disorders, especially skin disorders, that involve an inflammatory component such as psoriasis, allergic or contact dermatitis.

### Example 18

Sequences of the cDNA, mRNA and corresponding amino acids for the NK-1 receptor follow. Accession numbers are given for each sequence.

SHORT mRNA TRANSCRIPT

LOCUS NM_{—}015727 1268 bp mRNA PRI 28-APR-2000

DEFINITION Homo sapiens tachykinin receptor 1 (TACR1), transcript variant short, mRNA.

ACCESSION NM_{—}015727 VERSION NM_{—}015727.1 GI:7669545 KEYWORDS.

SOURCE human.

ORGANISM Homo sapiens Eukaryota; Metazoa; Chordata; Craniata; Vertebrata; Euteleostomi; Mammalia; Eutheria; Primates; Catarrhini; Hominidae; Homo.

COMMENT REFSEQ: This reference sequence was derived from M84426.1. Summary: This gene belongs to a family of genes that function as receptors for tachykinins. Receptor affinities are specified by variations in the 5′-end of the sequence. The receptors belonging to this family are characterized by interactions with G proteins and 7 hydrophobic transmembrane regions. This gene encodes the receptor for the tachykinin substance P, also referred to as neurokinin 1. This receptor is also involved in the mediation of phosphatidylinositol metabolism of substance P. Transcript Variant: Transcript variant short encodes a truncated polypeptide as compared to the full-length transcript variant long.

FEATURES Location/Qualifiers source 1.1268/organism=“Homo sapiens”/db_xref=“taxon:9606”/chromosome=“2”/map=“2” gene 1.1268/gene=“TACR1”/note=“NK1R; TAC1R; SPR”/db-xref-“LocusID:6869”/db_xref=“MIM:162323” CDS 123.1058/gene=“TACR1”/note=“Isoform short is encoded by transcript variant short; substance P receptor; neurokinin 1 receptor”/codon_start=1/product=“tachykinin receptor 1, isoform short”/protein_id=“NP 056542.1”/db_xref=“GI:7669546”

| | |
|---|---|
| translation = “MDNVLPVDSDLSPNISTNTSEPNQFVQPAWQIVLWAAAYTVIVV | (SEQ ID NO 1) |
| TSVVGNVVVMWIILAHKRMRTVTNYFLVNLAFAEASMAAFNTVVNFTYAVHNEW | |
| YYGL | |
| FYCKFHNFFPIAAVFASIYSMTAVAFDRYMAIIHPLQPRLSATATKVVICVIWVLALL | |
| LAFPQGYYSTTETMPSRVVCMIEWPEHPNKIYEKVYHICVTVLIYFLPLLVIGYAYTV | |
| VGITLWASEIPGDSSDRYHEQVSAKRKVVKMMIVVVCTFAICWLPFHIFFLLPYINPD | |
| LYLKKFIQQVYLAIMWLAMSSTMYNPIIYCCLNDR” | |

BASE COUNT 291 a 383 c 295 g 299 t
ORIGIN

| | | |
|---|---|---|
| 1 | gaaaaagcct tccaccctcc tgtctggctt tagaaggacc ctgagcccca ggcgccacga | (SEQ ID NO 2) |
| 61 | caggactctg ctgcagaggg gggttgtgta cagatagtag ggctttaccg cctagcttcg | |
| 121 | aaatggataa cgtcctcccg gtggactcag acctctcccc aaacatctcc actaacacct | |
| 181 | cggaacccaa tcagttcgtg caaccagcct ggcaaattgt cctttgggca gctgcctaca | |
| 241 | cggtcattgt ggtgacctct gtggtgggca acgtggtagt gatgtggatc atcttagccc | |
| 301 | acaaaagaat gaggacagtg acgaactatt ttctggtgaa cctggccttc gcggaggcct | |
| 361 | ccatggctgc attcaataca gtggtgaact tcacctatgc tgtccacaac gaatggtact | |
| 421 | acggcctgtt ctactgcaag ttccacaact tcttccccat cgccgctgtc ttcgccagta | |
| 481 | tctactccat gacggctgtg gcctttgata ggtacatggc catcatacat cccctccagc | |
| 541 | cccggctgtc agccacagcc accaaagtgg tcatctgtgt catctgggtc ctggctctcc | |
| 601 | tgctggcctt cccccagggc tactactcaa ccacagagac catgcccagc agagtcgtgt | |
| 661 | gcatgatcga atggccagag catccgaaca agatttatga gaaagtgtac cacatctgtg | |
| 721 | tgactgtgct gatctacttc ctccccctgc tggtgattgg ctatgcatac accgtagtgg | |
| 781 | gaatcacact atgggccagt gagatccccg gggactcctc tgaccgctac cacgagcaag | |
| 841 | tctctgccaa gcgcaaggtg gtcaaaatga tgattgtcgt ggtgtgcacc ttcgccatct | |
| 901 | gctggctgcc cttccacatc ttcttcctcc tgccctacat caacccagat ctctacctga | |
| 961 | agaagtttat ccagcaggtc tacctggcca tcatgtggct ggccatgagc tccaccatgt | |
| 1021 | acaaccccat catctactgc tgcctcaatg acaggtgagg atcccaaccc catgagctct | |
| 1081 | ccaggggcca caagaccatc tacatacaca gtggccaagc ggcatcctaa atgagtaaac | |
| 1141 | ccagctgtga gacaagaggg acaagtgggg actgcagcta acttatcatc acacaactca | |
| 1201 | gcctggctga ttatcaccat ccaggaatgg gagcccggag tagactgatt ttcttttttt | |
| 1261 | cttttcca | |

LOCUS HUMSPRSHOR 1268 bp mRNA PRI Aug. 3, 1993

DEFINITION Homo sapiens substance P receptor (short form) mRNA, complete cds.

ACCESSION M84426 VERSION M84426.1 GI:338435 KEYWORDS substance P receptor (short form).

SOURCE Homo sapiens brain cDNA to mRNA.

ORGANISM Homo sapiens Eukaryota; Metazoa; Chordata; Craniata; Vertebrata; Euteleostomi; Mammalia; Eutheria; Primates; Catarrhini; Hominidae; Homo.

| | |
|---|---|
| translation = “MDNVLPVDSDLSPNISTNTSEPNQFVQPAWQIVLWAAAYTVIVV | (SEQ ID NO 3) |
| TSVVGNVVVMWIILAHKRMRTVTNYFLVNLAFAEASMAAFNTVVNFTYAVHNEW | |
| YYGL | |
| FYCKFHNFFPIAAVFASIYSMTAVAFDRYMAIIHPLQPRLSATATKVVICVIWVLALL | |
| LAFPQGYYSTTETMPSRVVCMIEWPEHPNKIYEKVYHICVTVLIYFLPLLVIGYAYTV | |
| VGITLWASEIPGDSSDRYHEQVSAKRKVVKMMIVVVCTFAICWLPFHIFFLLPYINPD | |
| LYLKKFIQQVYLAIMWLAMSSTMYNPIIYCCLNDR” | |

BASE COUNT 291 a 383 c 295 g 299 t
ORIGIN

| | | |
|---|---|---|
| 1 | gaaaaagcct tccaccctcc tgtctggctt tagaaggacc ctgagcccca ggcgccacga | (SEQ ID NO 5) |
| 61 | caggactctg ctgcagaggg gggttgtgta cagatagtag ggctttaccg cctagcttcg | |
| 121 | aaatggataa cgtcctcccg gtggactcag acctctcccc aaacatctcc actaacacct | |
| 181 | cggaacccaa tcagttcgtg caaccagcct ggcaaattgt cctttgggca gctgcctaca | |
| 241 | cggtcattgt ggtgacctct gtggtgggca acgtggtagt gatgtggatc atcttagccc | |
| 301 | acaaaagaat gaggacagtg acgaactatt ttctggtgaa cctggccttc gcggaggcct | |
| 361 | ccatggctgc attcaataca gtggtgaact tcacctatgc tgtccacaac gaatggtact | |
| 421 | acggcctgtt ctactgcaag ttccacaact tcttccccat cgccgctgtc ttcgccagta | |
| 481 | tctactccat gacggctgtg gcctttgata ggtacatggc catcatacat cccctccagc | |
| 541 | cccggctgtc agccacagcc accaaagtgg tcatctgtgt catctgggtc ctggctctcc | |
| 601 | tgctggcctt cccccagggc tactactcaa ccacagagac catgcccagc agagtcgtgt | |
| 661 | gcatgatcga atggccagag catccgaaca agatttatga gaaagtgtac cacatctgtg | |
| 721 | tgactgtgct gatctacttc ctccccctgc tggtgattgg ctatgcatac accgtagtgg | |
| 781 | gaatcacact atgggccagt gagatccccg gggactcctc tgaccgctac cacgagcaag | |
| 841 | tctctgccaa gcgcaaggtg gtcaaaatga tgattgtcgt ggtgtgcacc ttcgccatct | |
| 901 | gctggctgcc cttccacatc ttcttcctcc tgccctacat caacccagat ctctacctga | |
| 961 | agaagtttat ccagcaggtc tacctggcca tcatgtggct ggccatgagc tccaccatgt | |
| 1021 | acaaccccat catctactgc tgcctcaatg acaggtgagg atcccaaccc catgagctct | |
| 1081 | ccaggggcca caagaccatc tacatacaca gtggccaagc ggcatcctaa atgagtaaac | |
| 1141 | ccagctgtga gacaagaggg acaagtgggg actgcagcta acttatcatc acacaactca | |
| 1201 | gcctggctga ttatcaccat ccaggaatgg gagcccggag tagactgatt ttcttttttt | |
| 1261 | cttttcca | |

LONG mRNA TRANSCRIPT

LOCUS NM_{—}001058 1766 bp mRNA PRI Apr. 28, 2000

DEFINITION Homo sapiens tachykinin receptor 1 (TACR1), transcript variant long, mRNA.

ACCESSION NM_{—}001058 VERSION NM_{—}001058.2 GI:7669544 KEYWORDS.

SOURCE human.

ORGANISM Homo sapiens Eukaryota; Metazoa; Chordata; Craniata; Vertebrata; Euteleostomi; Mammalia; Eutheria; Primates; Catarrhini; Hominidae; Homo.

COMMENT REFSEQ: This reference sequence was derived from M74290.1. On Apr. 28, 2000 this sequence version replaced gi:4507342. Summary: This gene belongs to a family of genes that function as receptors for tachykinins. Receptor affinities are specified by variations in the 5′-end of the sequence. The receptors belonging to this family are characterized by interactions with G proteins and 7 hydrophobic transmembrane regions. This gene encodes the receptor for the tachykinin substance P, also referred to as neurokinin 1. This receptor is also involved in the mediation of phosphatidylinositol metabolism of substance P. Transcript Variant: Transcript variant long represents the most complete and predominant form of this gene. FEATURES Location/Qualifiers source 1.1766/organism=“Homo sapiens”/db_xref=“taxon:9606”/chromosome=“2”/map=“2” gene 1.1766/gene=“TACR1”/note=“NK1R; TAC1R; SPR”/db_xref=“LocusID:6869”/db_xref=“MIM:162323” CDS 211.1434/gene=“TACR1”/note=“Isoform long is encoded by transcript variant long; substance P receptor; neurokinin 1 receptor”/codon_start=1/product=“tachykinin receptor 1, isoform long”/protein id=“NP 001049.1”/db_xref=“GI:4507343”

| | |
|---|---|
| translation = “MDNVLPVDSDLSPNISTNTSEPNQFVQPAWQIVLWAAAYTVIVV | (SEQ ID NO 5) |
| TSVVGNVVVMWIILAHKRMRTVTNYFLVNLAFAEASMAAFNTVVNFTYAVHNEW | |
| YYGL | |
| FYCKFHNFFPIAAVFASIYSMTAVAFDRYMAIIHPLQPRLSATATKVVICVIWVLALL | |
| LAFPQGYYSTTETMPSRVVCMIEWPEHPNKIYEKVYHICVTVLIYFLPLLVIGYAYTV | |
| VGITLWASEIPGDSSDRYHEQVSAKRKVVKMMIVVVCTFAICWLPFHIFFLLPYINPD | |
| LYLKKFIQQVYLAIMWLAMSSTMYNPIIYCCLNDRFRLGFKHAFRCCPFISAGDYEG | |
| L | |
| EMKSTRYLQTQGSVYKVSRLETTISTVVGAHEEEPEDGPKATPSSLDLTSNCSSRSDS | |
| KTMTESFSFSSNVLS” | |

BASE COUNT 401 a 540 c 412 g 413 t
ORIGIN

| | | |
|---|---|---|
| 1 | aattcagagc caccgcgggc aggcgggcag tgcatccaga agcgtttata ttctgagcgc | (SEQ ID NO 6) |
| 61 | cagttcagct ttcaaaaaga gtgctgccca taaaaagcct tccaccctcc tgtctgcttt | |
| 121 | agaaggaccc tgagccccag gcgccagcca caggactctg ctgcagaggg gggttgtgta | |
| 181 | cagatagtag gctttacgcc tagcttcgaa atggataacg tcctcccggt ggactcagac | |
| 241 | ctctccccaa acatctccac taacacctcg gaacccaatc agttcgtgca accagcctgg | |
| 301 | caaattgtcc tttgggcagc tgcctacacg gtcattgtgg tgacctctgt ggtgggcaac | |
| 361 | gtggtagtga tgtggatcat cttagcccac aaaagaatga ggacagtgac gaactatttt | |
| 421 | ctggtgaacc tggccttcgc ggaggcctcc atggctgcat tcaatacagt ggtgaacttc | |
| 481 | acctatgctg tccacaacga atggtactac ggcctgttct actgcaagtt ccacaacttc | |
| 541 | tttcccatcg ccgctgtctt cgccagtatc tactccatga cggctgtggc ctttgatagg | |
| 601 | tacatggcca tcatacatcc cctccagccc cggctgtcag ccacagccac caaagtggtc | |
| 661 | atctgtgtca tctgggtcct ggctctcctg ctggccttcc cccagggcta ctactcaacc | |
| 721 | acagagacca tgcccagcag agtcgtgtgc atgatcgaat ggccagagca tccgaacaag | |
| 781 | atttatgaga aagtgtacca catctgtgtg actgtgctga tctacttcct ccccctgctg | |
| 841 | gtgattggct atgcatacac cgtagtggga atcacactat gggccagtga gatccccggg | |
| 901 | gactcctctg accgctacca cgagcaagtc tctgccaagc gcaaggtggt caaaatgatg | |
| 961 | attgtcgtgg tgtgcacctt cgccatctgc tggctgccct tccacatctt cttcctcctg | |
| 1021 | ccctacatca acccagatct ctacctgaag aagtttatcc agcaggtcta cctggccatc | |
| 1081 | atgtggctgg ccatgagctc caccatgtac aaccccatca tctactgctg cctcaatgac | |
| 1141 | aggttccgtc tgggcttcaa gcatgccttc cggtgctgcc ccttcatcag cgccggcgac | |
| 1201 | tatgaggggc tggaaatgaa atccacccgg tatctccaga cccagggcag tgtgtacaaa | |
| 1261 | gtcagccgcc tggagaccac catctccaca gtggtggggg cccacgagga ggagccagag | |
| 1321 | gacggcccca aggccacacc ctcgtccctg gacctgacct ccaactgctc ttcacgaagt | |
| 1381 | gactccaaga ccatgacaga gagcttcagc ttctcctcca atgtgctctc ctaggccaca | |
| 1441 | gggcctttgg caggtgcagc ccccactgcc tttgacctgc ctcccttcat gcatggaaat | |
| 1501 | tcccttcatc tggaaccatc agaaacaccc tcacactggg acttgcaaaa agggtcagta | |
| 1561 | tgggttaggg aaaacattcc atccttgagt caaaaaatct caattcttcc ctatctttgc | |
| 1621 | caccctcatg ctgtgtgact caaaccaaat cactgaactt tgctgagcct gtaaaataaa | |
| 1681 | aggtcggacc agcttttcct caagagccca atgcattcca tttctggaag tgactttggc | |
| 1741 | tgcatgcgag tgctcatttc aggatg | |

COMPLETE CDs

LOCUS HUMNKIRX 1230 bp mRNA PRI Jan. 7, 1995

DEFINITION Human neurokinin 1 receptor (NK1R) mRNA, complete cds.

ACCESSION M76675 VERSION M76675.1 GI:189231 KEYWORDS GTP-binding protein; neurokinin 1 receptor; neurotransmitter receptor; plasma membrane protein; protein coupled; substance P receptor.

SOURCE Homo sapiens lymphocyte cDNA to mRNA.

ORGANISM Homo sapiens Eukaryota; Metazoa; Chordata; Craniata; Vertebrata; Euteleostomi; Mammalia; Eutheria; Primates; Catarrhini; Hominidae; Homo.

| | |
|---|---|
| translation = “MDNVLPVDSDLSPNISTNTSEPNQFVQPAWQIVLWAAAYTVIVV | (SEQ ID NO 7) |
| TSVVGNVVVMWIILAHKRMRTVTNYFLVNLAFAEASMAAFNTVVNFTYAVHNEW | |
| YYGL | |
| FYCKFHNFFPIAACFASIYSMTAVAFDRYMAIIHPLQPRLSATATKVVICVIWVLALL | |
| LAFPQGYYSTTETMPSRVVCMIEWPEHPNKIYEKVYHICVTVLIYFLPLLVIGYAYTI | |
| VGITLWASEIPGDSSDRYHEQVSAKRKVVKMMIVVVCTFAICWLPFHIFFLLPYINPD | |
| LYLKKFIQQVYLAIMWLAMSSTMYNPIIYCCLNDRFRLGFKHAFRCCPFISAGDYEG | |
| L | |
| EMKSTRYLQTQGSVYKVSRLETTISTVVGAHEEEPEDGPKATPSSLDLTSNCSSRSDS | |
| KTMTESFSFSSNVLS” | |

BASE COUNT 268 a 390 c 289 g 283 t
ORIGIN chromosome 2.

| | | |
|---|---|---|
| 1 | atggataacg tcctcccggt ggactcagac ctctccccaa acatctccac taacacctcg | (SEQ ID NO 8) |
| 61 | gaacccaatc agttcgtgca accagcctgg caaattgtcc tttgggcagc tgcttacacg | |
| 121 | gtcattgtgg tgacctctgt ggtgggcaac gtggtagtga tgtggatcat cttagcccac | |
| 181 | aaaagaatga ggacagtgac gaactatttt ctggtgaacc tggccttcgc ggaggcctcc | |
| 241 | atggctgcat tcaatacagt ggtgaacttc acctatgctg tccacaacga atggtactac | |
| 301 | ggcctgttct actgcaagtt ccacaacttc ttccccatcg ccgcttgctt cgccagtatc | |
| 361 | tactccatga cggctgtggc ctttgatagg tacatggcca tcatacatcc cctccagccc | |
| 421 | cggctgtcag ccacagccac caaagtggtc atctgtgtca tctgggtcct ggctctcctg | |
| 481 | ctggccttcc cccagggcta ctactcaacc acagagacca tgcccagcag agtcgtgtgc | |
| 541 | atgatcgaat ggccagagca tccgaacaag atttatgaga aagtgtacca catctgtgtg | |
| 601 | actgtgctga tctacttcct ccccctgctg gtgattggct atgcatacac catagtggga | |
| 661 | atcacactat gggccagtga gatccccggg gactcctctg accgctacca cgagcaagtc | |
| 721 | tctgccaagc gcaaggtggt caaaatgatg attgtcgtgg tgtgcacctt cgccatctgc | |
| 781 | tggctgccct tccacatctt cttcctcctg ccctacatca acccagatct ctacctgaag | |
| 841 | aagtttatcc agcaggtcta cctggccatc atgtggctgg ccatgagctc caccatgtac | |
| 901 | aaccccatca tctactgctg cctcaatgac aggttccgtc tgggcttcaa gcatgccttc | |
| 961 | cggtgctgcc ccttcatcag cgccggcgac tatgaggggc tggaaatgaa atccacccgg | |
| 1021 | tatctccaga cccagggcag tgtgtacaaa gtcagccgcc tggagaccac catctccaca | |
| 1081 | gtggtggggg cccacgagga ggagccagag gacggcccca aggccacacc ctcgtccctg | |
| 1141 | gacctgacct ccaactgctc ttcacgaagt gactccaaga ccatgacaga gagcttcagc | |
| 1201 | ttctcctcca atgtgctctc ctagggatcc | |

| | |
|---|---|
| SEQ ID NO 9 | 5′ GAC GTT ATC CAT TTT GGG GCA 3′ |
| SEQ ID NO 10 | 5′ GAC GTT ATC CAT TTT GGG GC 3′ |
| SEQ ID NO 11 | 5′ GAC GTT ATC CAT TTT GGG G 3′ |
| SEQ ID NO 12 | 5′ GAC GTT ATC CAT TTT GGG 3′ |
| SEQ ID NO 13 | 5′ GAC GTT ATC CAT TTT GG 3′ |
| SEQ ID NO 14 | 5′ GAC GTT ATC CAT TTT G 3′ |
| SEQ ID NO 15 | 5′ GAC GTT ATC CAT TTT 3′ |
| SEQ ID NO 16 | 5′ AC GTT ATC CAT TTT GGG GCA 3′ |
| SEQ ID NO 17 | 5′ C GTT ATC CAT TTT GGG GCA 3′ |
| SEQ ID NO 18 | 5′ GTT ATC CAT TTT GGG GCA 3′ |
| SEQ ID NO 19 | 5′ TT ATC CAT TTT GGG GCA 3′ |
| SEQ ID NO 20 | 5′ T ATC CAT TTT GGG GCA 3′ |
| SEQ ID NO 21 | 5′ ATC CAT TTT GGG GCA 3′ |

| Sense | | Antisense | |
|---|---|---|---|
| ttc cac atc ttc ttc ctc ct | (SEQ ID NO 41) | agg agg aag aag atg tgg | (SEQ ID NO 60) |
| | | aa | |
| tga tga ttg tcg tgg tgt gca | (SEQ ID NO 42) | tgc aca cca cga caa tca | (SEQ ID NO 61) |
| | | tca | |
| gca agt ctc tgc caa gcg | (SEQ ID NO 43) | ttg cgc ttg gca gag act | (SEQ ID NO 62) |
| caa | | tgc | |
| ttg atg tag ggc agg agg aa | (SEQ ID NO 44) | ttc ctc ctg ccc tac atc aa | (SEQ ID NO 63) |
| tgc aca cca cga caa tca tca | (SEQ ID NO 45) | tga tga ttg tcg tgg tgt | (SEQ ID NO 64) |
| | | gca | |
| cat agt gtg att ccc act ac | (SEQ ID NO 46) | gta gtg gga atc aca cta | (SEQ ID NO 65) |
| | | tg | |
| atg cat agc caa tca cca gca | (SEQ ID NO 47) | tgc tgg tga ttg gct atg | (SEQ ID NO 66) |
| | | cat | |
| act ttg gtg gct gtg gct ga | (SEQ ID NO 48) | tca gcc aca gcc acc aaa | (SEQ ID NO 67) |
| | | gt | |
| gga tgt atg atg gcc atg ta | (SEQ ID NO 49) | tac atg gcc atc ata cat cc | (SEQ ID NO 68) |
| cat gga gta gat act ggc | (SEQ ID NO 50) | ttc gcc agt atc tac tcc | (SEQ ID NO 69) |
| gaa | | atg | |
| gaa gaa gtt gtg gaa ctt gca | (SEQ ID NO 51) | tgc aag ttc cac aac ttc | (SEQ ID NO 70) |
| | | ttc | |
| gta gac ctg ctg gat aaa ctt | (SEQ ID NO 52) | aag ttt atc cag cag gtc | (SEQ ID NO 71) |
| | | tac | |
| aca gta gat gat ggg gtt gta | (SEQ ID NO 53) | atg tac aac ccc atc atc | (SEQ ID NO 72) |
| cat | | tac | |
| gtg tac aga tag tag gct t | (SEQ ID NO 54) | aag cct act atc tgt aca c | (SEQ ID NO 73) |
| cct cct gtc tgg ctt tag aa | (SEQ ID NO 55) | ttc taa agc cag aca gga | (SEQ ID NO 74) |
| | | gg | |
| aac cca tac tga ccc ttt t | (SEQ ID NO 56) | aaa agg gtc agt atg ggt t | (SEQ ID NO 75) |
| caa gga tgg aat gtt ttc cct | (SEQ ID NO 57) | agg gaa aac att cca tcc | (SEQ ID NO 76) |
| | | ttg | |
| tct cta cct gaa gaa gtt | (SEQ ID NO 58) | aac ttc ttc agg tag aga | (SEQ ID NO 77) |
| ttc gaa atg gat aac gtc ctc | (SEQ ID NO 59) | gag gac gtt atc cat ttc | (SEQ ID NO 78) |
| | | gaa | |

What is claimed is:

1. A method of treating a pathological condition characterized at least partially by involvement of the NK-1 receptor, said method comprising,
administering to a mammal in need thereof, a therapeutically effective amount of at least one oligonucleotide or oligonucleotide analog which interferes with the function or production of NK-1 receptors.

2. The method of claim 1, wherein said interference with said function or production of said NK-1 receptors involves at least one nucleic acid in the NK-1 receptor pathway.

3. The method of claim 2, wherein said nucleic acid is one or more selected from the group consisting of DNA, RNA, tRNA, mRNA and rRNA.

4. The method of claim 1, wherein said oligonucleotide comprises RNA in the form of at least one ribozyme.

5. The method of claim 1, wherein said oligonucleotide or oligonucleotide analog is one or more selected from oligonucleotides and oligonucleotide analogs that are complementary to nucleic acid in said NK-1 receptor pathway.

6. The method of claim 1, wherein said oligonucleotide or oligonucleotide analog is one or more selected from the group consisting of DNA antisense oligonucleotides and oligonucleotide analogs, RNA antisense oligonucleotides and oligonucleotide analogs, DNA sense oligonucleotides and oligonucleotide analogs, RNA sense oligonucleotides and oligonucleotide analogs, aptamers and ribozymes.

7. The method of claim 1, wherein said oligonucleotide or oligonucleotide analog is at least one selected from those that are complementary to at least a portion of the NK-1 receptor DNA or RNA shown in SEQ. ID Nos. 2, 4, 6 and 8.

8. The method of claim 1, wherein said oligonucleotide or oligonucleotide analog is one or more selected from the group consisting of those shown in SEQ. ID Nos. 9-59.

9. The method of claim 1, wherein said mammal is a human.

10. The method of claim 1, wherein said oligonucleotide or oligonucleotide analog is applied by intrathecal infusion to the spinal canal.

11. The method of claim 1, wherein the amount of said oligonucleotide or oligonucleotide analog that is administered is from 15 to 30 nanomoles per kilogram of body weight of said mammal.

12. The method of claim 1, wherein the amount of said oligonucleotide or oligonucleotide analog that is administered is from 20 to 25 nanomoles per kilogram of body weight of said mammal.

13. The method of claim 1, wherein the amount of said oligonucleotide or oligonucleotide analog that is administered is from 15 to 300 nanomoles per kilogram of body weight of said mammal.

14. The method of claim 1, wherein the amount of said oligonucleotide or oligonucleotide analog that is administered is from 50 to 600 micrograms per kilogram of body weight of said mammal.

15. The method of claim 1, wherein the amount of said oligonucleotide or oligonucleotide analog that is administered is from 200 to 400 micrograms per kilogram of body weight of said mammal.

16. The method of claim 1, wherein the amount of said oligonucleotide or oligonucleotide analog that is administered is from 250 to 350 micrograms per kilogram of body weight of said mammal.

17. The method of claim 1, wherein said oligonucleotide or oligonucleotide analog is administered via intravenous infusion.

18. The method of claim 1, wherein said oligonucleotide or oligonucleotide analog is administered by one or more routes selected from the group consisting of oral, parenteral, rectal, sub-cutaneous, mucosal, buccal, transdermal, intravaginal, nasal, nasal inhalation, pulmonary inhalation, iontophoresis through the skin, iontophoresis through mucosal or buccal membranes, dermal patch, epidural, intracranial, intrapharyngeal, sublingual, intra-articular, intramuscular, and subcutaneous.

19. The method of claim 1, wherein said pathological condition is one or more selected from the group consisting of dermatological disorders, immune disorders, autoimmune disorders, cardiovascular disorders, vascular disorders, gut inflammation, arthritis, airway disorders, neuropathic disorders, central aspects of chronic or acute pain, peripheral aspects of chronic or acute pain, psychiatric disorders, and central nervous system disorders.

20. The method of claim 19, wherein said vascular disorder is migraine.

21. The method of claim 19, wherein said nervous system disorder is at least one selected from the group consisting of anxiety, psychosis, and schizophrenia.

22. A pharmaceutical preparation comprising at least one oligonucleotide or oligonucleotide analog selected from the group consisting of oligonucleotides and oligonucleotide analogs that interfere with the function or production of at least a portion of said NK-1 receptor.

23. The pharmaceutical preparation of claim 22, in admixture with at least one pharmaceutically acceptable substance from the group consisting of excipients, penetration enhancers, stabilizers, absorption enhancers and carrier compounds.

24. The pharmaceutical preparation of claim 22, wherein said oligonucleotide or oligonucleotide analog is one or more selected from the group consisting of those shown in SEQ. ID Nos. 9-59.

25. The pharmaceutical preparation of claim 22, wherein said oligonucleotide or oligonucleotide analog is complementary to any nucleic acid in said NK-1 receptor pathway.

26. The pharmaceutical preparation of claim 25, wherein said nucleic acid in said NK-1 receptor pathway is RNA.

27. The pharmaceutical preparation of claim 22, wherein said nucleic acid in said NK-1 receptor pathway is DNA.

28. The pharmaceutical preparation of claim 22, wherein said preparation is administered to treat one or more pathological conditions selected from the group consisting of dermatological disorders, immune disorders, autoimmune disorders, neuropathic disorders, cardiovascular disorders, vascular disorders, gut inflammation, arthritis, airway disorders, central aspects of chronic or acute pain, peripheral aspects of chronic or acute pain, psychiatric disorders, and central nervous system disorders.

29. The pharmaceutical preparation of claim 26, wherein said vascular disorder is migraine.

30. The pharmaceutical preparation of claim 26, wherein said nervous system disorder is at least one selected from the group consisting of anxiety, psychosis, and schizophrenia.

31. A kit for treating or diagnosing a pathological condition, said kit comprising a pharmaceutical preparation comprising at least one oligonucleotide or oligonucleotide analog selected from the group consisting of oligonucleotides and oligonucleotide analogs that interfere with the function or production of at least a portion of said NK-1 receptor, and instructions for administering said pharmaceutical preparation to a mammal.

32. The kit of claim 31, wherein said pathological condition is one or more selected from the group consisting of dermatological disorders, immune disorders, autoimmune disorders, neuropathic disorders, cardiovascular disorders, vascular disorders, gut inflammation, arthritis, airway disorders, central aspects of chronic or acute pain, peripheral aspects of chronic or acute pain, psychiatric disorders, and central nervous system disorders.

33. The kit of claim 31, wherein said at least one oligonucleotide or oligonucleotide analog is in admixture with at least one pharmaceutically acceptable substance from the group consisting of excipients, penetration enhancers, stabilizers, absorption enhancers and carrier compounds.

34. A method of treating, attenuating or preventing pain comprising, administering to a mammal in need thereof, a therapeutically effective amount of at least one compound that interferes with the function or production of NK-1 receptors.

35. The method of claim 34, wherein said compound is at least one selected from the group consisting of oligonucleotides or oligonucleotide analogs, and non-nucleotide disruptor compounds.

36. The method of claim 34, wherein said interference with the function or production of said NK-1 receptors involves at least one nucleic acid in the NK-1 receptor pathway.

37. The method of claim 36, wherein said nucleic acid is one or more selected from the group consisting of DNA, RNA, tRNA, mRNA and rRNA.

38. The method of claim 35, wherein said oligonucleotide comprises RNA in the form of at least one ribozyme.

39. The method of claim 35, wherein said oligonucleotide or oligonucleotide analog is one or more selected from oligonucleotide or oligonucleotide analogs that are complementary to nucleic acid in said NK-1 receptor pathway.

40. The method of claim 35, wherein said oligonucleotide or oligonucleotide analog is one or more selected from the group consisting of DNA antisense oligonucleotides or oligonucleotide analogs, RNA antisense oligonucleotides or oligonucleotide analogs, DNA sense oligonucleotides or oligonucleotide analogs, RNA sense oligonucleotides or oligonucleotide analogs, aptamers and ribozymes.

41. The method of claim 35, wherein said oligonucleotide or oligonucleotide analog is one or more selected from the group consisting of those shown in SEQ. ID. Nos. 9-59.

42. The method of claim 34, wherein said mammal is a human.

43. The method of claim 35, wherein said disruptor is one or more selected from the group consisting of methylation compounds, de-methylation compounds, antibodies to nucleic acids, mutagens, intercalation compounds, gyrases, ligases, and methylases.

44. The method of claim 34, wherein said compound is applied by intrathecal infusion to the spinal canal.

45. The method of claim 34, wherein said compound is administered by one or more routes selected from the group consisting of oral, buccal, mucosal, parenteral, rectal, sub-cutaneous, transdermal, intravaginal, nasal, nasal inhalation, pulmonary inhalation, iontophoresis through the skin, iontophoresis through mucosal or buccal membranes, dermal patch, epidural, intracranial, intrapharyngeal, sublingual, intra-articular, intramuscular, and subcutaneous.

46. The method of claim 34, wherein the amount of said compound that is administered is from 15 to 30 nanomoles per kilogram of body weight of said mammal.

47. The method of claim 34, wherein the amount of said compound that is administered is from 20 to 25 nanomoles per kilogram of body weight of said mammal.

48. The method of claim 34, wherein the amount of said compound that is administered is from 15 to 30 nanomoles per kilogram of body weight of said mammal.

49. The method of claim 34, wherein the amount of said compound that is administered is from 50 to 600 micrograms per kilogram of body weight of said mammal.

50. The method of claim 34, wherein the amount of said compound that is administered is from 200 to 400 micrograms per kilogram of body weight of said mammal.

51. The method of claim 34, wherein the amount of said compound that is administered is from 250 to 350 micrograms per kilogram of body weight of said mammal.

52. The method of claim 34, wherein said compound is administered via intravenous infusion.

53. The method of claim 34, wherein said pain is characterized as peripheral pain, chronic pain, acute pain, neuropathic pain, pain relating to psychiatric disorders, and pain relating to central nervous system disorders.

54. The method of claim 34, wherein said pain is chronic pain.

55. The method of claim 34, wherein said pain is neuropathic pain.

56. The method of claim 34, wherein said pain is characterized by at least one from the group consisting of hyperalgesia, allodynia, neuralgia, and dysesthesia.

57. The method of claim 35, wherein said non-nucleotide disruptor acts directly upon nucleic acid in said NK-1 pathway.

58. The method of claim 34, wherein said non-nucleotide disruptor does not act directly upon nucleic acid in said NK-1 pathway.

59. A pharmaceutical preparation useful for preventing, attenuating or treating pain, comprising at least one compound selected from the group consisting of compounds that interfere with the function or production of NK-1 receptors.

60. The pharmaceutical preparation of claim 59, wherein said compound is at least one selected from the group consisting of oligonucleotides or oligonucleotide analogs, and non-nucleotide disruptor compounds.

61. The pharmaceutical preparation of claim 59, in admixture with at least one pharmaceutically acceptable substance from the group consisting of excipients, penetration enhancers, stabilizers, absorption enhancers and carrier compounds.

62. The pharmaceutical preparation of claim 59, wherein said oligonucleotide or oligonucleotide analog is complementary to any nucleic acid in said NK-1 receptor pathway.

63. The pharmaceutical preparation of claim 59, wherein said oligonucleotide or oligonucleotide analog is at least one selected from those that are complementary to at least a portion of the NK-1 receptor DNA or RNA of those shown in SEQ. ID Nos. 2, 4, 6 and 8.

64. The pharmaceutical of claim 59, wherein said disruptor is one or more selected from the group consisting of methylation compounds, de-methylation compounds, antibodies to nucleic acids, mutagens, intercalation compounds, gyrases, ligases, and methylases.

65. A kit for treating, diagnosing, attenuating or preventing pain, said kit comprising a pharmaceutical preparation comprising at least one compound selected from the group consisting of oligonucleotides and oligonucleotide analogs, and non-nucleotide disruptors that interfere with the function or production of at least a portion of the NK-1 receptor, and instructions for administering said compound to a mammal.

66. The kit of claim 65, wherein said pain is characterized as peripheral pain, chronic pain, acute pain neuropathic pain, pain relating to psychiatric disorders, and pain relating to central nervous system disorders.

67. The kit of claim 65, wherein said pain is characterized by at least one from the group consisting of hyperalgesia, allodynia, neuralgia, and dysesthesia.

68. The kit of claim 65, wherein said compound is in admixture with at least one pharmaceutically acceptable substance from the group consisting of excipients, penetration enhancers, stabilizers, absorption enhancers and carrier compounds.

69. A method of treating, attenuating or preventing an inflammatory condition characterized at least partially by activation of the NK-1 receptor comprising, administering to a mammal in need thereof, a therapeutically effective amount of at least one compound that interferes with the function or production of NK-1 receptors.

70. The method of claim 69, wherein said compound is at least one selected from the group consisting of oligonucleotides or oligonucleotide analogs and non-nucleotide disrupter compounds.

71. The method of claim 69, wherein said interference with the function or production of said NK-1 receptors involves at least one nucleic acid in the NK-1 receptor pathway.

72. The method of claim 69, wherein said nucleic acid is one or more selected from the group consisting of DNA, RNA, tRNA, mRNA and rRNA.

73. The method of claim 69, wherein said oligonucleotide comprises RNA in the form of at least one ribozyme.

74. The method of claim 70, wherein said oligonucleotide or oligonucleotide analog is one or more selected from oligonucleotide or oligonucleotide analogs that are complementary to nucleic acid in said NK-1 receptor pathway.

75. The method of claim 70, wherein said oligonucleotide or oligonucleotide analog is one or more selected from the group consisting of DNA antisense oligonucleotide or oligonucleotide analogs, RNA antisense oligonucleotide or oligonucleotide analogs, DNA sense oligonucleotide or oligonucleotide analogs, RNA sense oligonucleotide or oligonucleotide analogs, aptamers and ribozymes.

76. The method of claim 70, wherein said oligonucleotide or oligonucleotide analog is one or more selected from the group consisting of those shown in SEQ. ID Nos. 9-59.

77. The method of claim 69, wherein said mammal is a human.

78. The method of claim 70, wherein said oligonucleotide or oligonucleotide analog is applied by intrathecal infusion to the spinal canal.

79. The method of claim 69, wherein said compound is administered by one or more routes selected from the group consisting of oral, mucosal, buccal, parenteral, rectal, sub-cutaneous, transdermal, intravaginal, nasal, nasal inhalation, pulmonary inhalation, iontophoresis through the skin, iontophoresis through mucosal or buccal membranes, dermal patch, epidural, intracranial, intrapharyngeal, sublingual, intra-articular, intramuscular, and subcutaneous.

80. The method of claim 69, wherein the amount of said compound that is administered is from 15 to 30 nanomoles per kilogram of body weight of said mammal.

81. The method of claim 69, wherein the amount of said compound that is administered is from 20 to 25 nanomoles per kilogram of body weight of said mammal.

82. The method of claim 69, wherein the amount of said compound that is administered is from 15 to 30 nanomoles per kilogram of body weight of said mammal.

83. The method of claim 69, wherein the amount of said compound that is administered is from 50 to 600 micrograms per kilogram of body weight of said mammal.

84. The method of claim 69, wherein the amount of said compound that is administered is from 200 to 400 micrograms per kilogram of body weight of said mammal.

85. The method of claim 69, wherein the amount of said compound that is administered is from 250 to 350 micrograms per kilogram of body weight of said mammal.

86. The method of claim 69, wherein said compound is administered via intravenous infusion.

87. The method of claim 69, wherein said inflammation is characterized as peripheral inflammation, chronic inflammation, acute inflammation, inflammation relating to psychiatric disorders, and inflammation relating to central nervous system disorders.

88. The method of claim 69, wherein said inflammation is chronic inflammation.

89. The method of claim 69, wherein said inflammation is neuropathic inflammation.

90. The method of claim 69, wherein said inflammation is characterized by at least one from the group consisting of hyperalgesia, allodynia, neuralgia, and dysesthesia.

91. The method of claim 70, wherein said non-nucleotide disrupter is one or more selected from the group consisting of methylation compounds, de-methylation compounds, antibodies to nucleic acids, mutagens, intercalation compounds, gyrases, ligases, and methylases.

92. The method of claim 70, wherein said non-nucleotide disrupter acts directly upon nucleic acid in said NK-1 pathway.

93. The method of claim 70, wherein said non-nucleotide disruptor does not act directly upon nucleic acid in said NK-1 pathway.

94. A pharmaceutical preparation useful for treating inflammation, comprising at least one compound selected from the group consisting of compounds that interfere with the function or production of NK-1 receptors.

95. The pharmaceutical preparation of claim 78, wherein said compound is at least one selected from the group consisting of oligonucleotides or oligonucleotide analogs and non-nucleotide disrupter compounds.

96. The pharmaceutical preparation of claim 78, in admixture with at least one pharmaceutically acceptable substance from the group consisting of excipients, penetration enhancers, stabilizers, absorption enhancers and carrier compounds.

97. The pharmaceutical preparation of claim 78, wherein said oligonucleotide or oligonucleotide analog is one or more selected from the group consisting of those shown in SEQ. ID Nos. 9-59.

98. The pharmaceutical preparation of claim 78, wherein said oligonucleotide or oligonucleotide analog is complementary to any nucleic acid in said NK-1 receptor pathway.

99. The pharmaceutical preparation of claim 78, wherein said oligonucleotide or oligonucleotide analog is at least one selected from those that are complementary to at least a portion of the NK-1 receptor DNA or RNA of those shown in SEQ. ID Nos. 2, 4, 6 and 8.

100. A kit for treating, diagnosing, attenuating or preventing inflammation, said kit comprising a pharmaceutical preparation comprising at least one compound selected from the group consisting of oligonucleotides and oligonucleotide analogs, and non-nucleotide disruptors that interfere with the function or production of at least a portion of the NK-1 receptor, and instructions for administering said compound to a mammal.

101. The kit of claim 100, wherein said inflammation is characterized as peripheral inflammation, chronic inflammation, acute inflammation, dermatological inflammation, neuropathic inflammation, inflammation relating to psychiatric disorders, and inflammation relating to central nervous system disorders.

102. The kit of claim 100, wherein said inflammation is characterized by at least one from the group consisting of hyperalgesia, allodynia, neuralgia, and dysesthesia.

103. The kit of claim 100, wherein said compound is in admixture with at least one pharmaceutically acceptable substance from the group consisting of excipients, penetration enhancers, stabilizers, absorption enhancers and carrier compounds.

104. The kit of claim 100, wherein said compound is a is one or more disruptors selected from the group consisting of methylation compounds, de-methylation compounds, antibodies, mutagens, intercalation compounds, gyrases, ligases, and methylases

105. A method of treating a pathological condition characterized at least partially by involvement of the NK-1 receptor, said method comprising,
administering to a mammal in need thereof, a therapeutically effective amount of at least one disrupter that interferes with the function or production of NK-1 receptors.

106. The method of claim 105, wherein said interference with said function or production of said NK-1 receptors involves at least one nucleic acid in the NK-1 receptor pathway.

107. The method of claim 105, wherein said disruptor is not a nucleic acid or nucleic acid analog.

108. The method of claim 105, wherein said disruptor is one or more selected from the group consisting of methylation compounds, de-methylation compounds, antibodies to nucleic acids, mutagens, intercalation compounds, gyrases, ligases, and methylases.

109. The method of claim 105, wherein said NK-1 disruptor acts directly upon nucleic acid in said NK-1 pathway.

110. The method of claim 105, wherein said NK-1 disruptor does not act directly upon nucleic acid in said NK-1 pathway.

111. The method of claim 106, wherein said nucleic acid is one or more selected from the group consisting of DNA, RNA, tRNA, mRNA and rRNA.

112. The method of claim 111, wherein said RNA is at least one ribozyme.

113. The method of claim 105, wherein said mammal is a human.

114. The method of claim 105, wherein said disruptor is applied by intrathecal infusion to the spinal canal.

115. The method of claim 105, wherein said disruptor is administered via intravenous infusion.

116. The method of claim 105, wherein said disruptor is administered by one or more routes selected from the group consisting of oral, parenteral, rectal, sub-cutaneous, transdermal, intravaginal, nasal, nasal inhalation, pulmonary inhalation, iontophoresis through the skin, iontophoresis through mucosal or buccal membranes, dermal patch, epidural, intracranial, intrapharyngeal, sublingual, intra-articular, intramuscular, and subcutaneous.

117. The method of claim 105, wherein the amount of said disrupter that is administered is from 15 to 30 nanomoles per kilogram of body weight of said mammal.

118. The method of claim 105, wherein the amount of said disruptor that is administered is from 20 to 25 nanomoles per kilogram of body weight of said mammal.

119. The method of claim 105, wherein the amount of said disruptor that is administered is from 15 to 30 nanomoles per kilogram of body weight of said mammal.

120. The method of claim 105, wherein the amount of said disrupter that is administered is from 50 to 600 micrograms per kilogram of body weight of said mammal.

121. The method of claim 105, wherein the amount of said disruptor that is administered is from 200 to 400 micrograms per kilogram of body weight of said mammal.

122. The method of claim 105, wherein the amount of said disruptor that is administered is from 250 to 350 micrograms per kilogram of body weight of said mammal.

123. The method of claim 105, wherein said pathological condition is one or more selected from the group consisting of dermatological disorders, autoimmune disorders, cardiovascular disorders, vascular disorders, gut inflammation, arthritis, airway disorders, central aspects of chronic or acute pain, peripheral aspects of chronic or acute pain, psychiatric disorders, and central nervous system disorders.

124. The method of claim 105, wherein said vascular disorder is migraine.

125. The method of claim 105, wherein said nervous system disorder is at least one selected from the group consisting of anxiety, psychosis, and schizophrenia.

126. A pharmaceutical preparation comprising at least one disruptor selected from the group consisting of compounds that interfere with the function or production of at least a portion of an NK-1 receptor.

127. The pharmaceutical preparation of claim 126, in admixture with at least one pharmaceutically acceptable substance from the group consisting of excipients, penetration enhancers, stabilizers, absorption enhancers and carrier compounds.

128. The pharmaceutical preparation of claim 126, wherein said disruptor acts upon RNA in said NK-1 receptor pathway.

129. The pharmaceutical preparation of claim 126, wherein said disruptor acts upon DNA in said NK-1 receptor pathway.

130. The pharmaceutical preparation of claim 126, wherein said disruptor acts upon at least one protein in said NK-1 receptor pathway.

131. The pharmaceutical preparation of claim 126, wherein said preparation is administered to treat one or more pathological conditions selected from the group consisting of dermatological disorders, autoimmune disorders, cardiovascular disorders, vascular disorders, gut inflammation, arthritis, airway disorders, central aspects of chronic or acute pain, peripheral aspects of chronic or acute pain, psychiatric disorders, and central nervous system disorders.

132. The pharmaceutical preparation of claim 126, wherein said vascular disorder is migraine.

133. The pharmaceutical preparation of claim 126, wherein said nervous system disorder is at least one selected from the group consisting of anxiety, psychosis, and schizophrenia.

134. A kit for treating or diagnosing a pathological condition, said kit comprising a pharmaceutical preparation comprising at least one disrupter selected from the group of compounds that interfere with the function or production of at least a portion of said NK-1 receptor, and instructions for administering said disruptor to a mammal.

135. The kit of claim 134, wherein said disruptor is one or more selected from the group consisting of methylation compounds, de-methylation compounds, antibodies to nucleic acids, mutagens, intercalation compounds, gyrases, ligases, and methylases.

136. The kit of claim 134, wherein said pathological condition is one or more selected from the group consisting of dermatological disorders, autoimmune disorders, cardiovascular disorders, vascular disorders, gut inflammation, arthritis, airway disorders, central aspects of chronic or acute pain, peripheral aspects of chronic or acute pain, psychiatric disorders, and central nervous system disorders.

137. The kit of claim 134, wherein said disrupter is in admixture with at least one pharmaceutically acceptable substance from the group consisting of excipients, penetration enhancers, stabilizers, absorption enhancers and carrier compounds.


SEQUENCE LISTING (59 sequences)

SEQ ID NO: 1 - protein, 311 aa, Homo sapiens
MDNVLPVDSDLSPNISTNTSEPNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVICVIWVLALLLAFPQGYYSTTETMPSRVVCMIEWPEHPNKIYEKVYHICVTVLIYFLPLLVIGYAYTVVGITLWASEIPGDSSDRYHEQVSAKRKVVKMMIVVVCTFAICWLPFHIFFLLPYINPDLYLKKFIQQVYLAIMWLAMSSTMYNPIIYCCLNDR

SEQ ID NO: 2 - DNA, 1268 bp, Homo sapiens
gaaaaagccttccaccctcctgtctggctttagaaggaccctgagccccaggcgccacgacaggactctgctgcagaggggggttgtgtacagatagtagggctttaccgcctagcttcgaaatggataacgtcctcccggtggactcagacctctccccaaacatctccactaacacctcggaacccaatcagttcgtgcaaccagcctggcaaattgtcctttgggcagctgcctacacggtcattgtggtgacctctgtggtgggcaacgtggtagtgatgtggatcatcttagcccacaaaagaatgaggacagtgacgaactattttctggtgaacctggccttcgcggaggcctccatggctgcattcaatacagtggtgaacttcacctatgctgtccacaacgaatggtactacggcctgttctactgcaagttccacaacttcttccccatcgccgctgtcttcgccagtatctactccatgacggctgtggcctttgataggtacatggccatcatacatcccctccagccccggctgtcagccacagccaccaaagtggtcatctgtgtcatctgggtcctggctctcctgctggccttcccccagggctactactcaaccacagagaccatgcccagcagagtcgtgtgcatgatcgaatggccagagcatccgaacaagatttatgagaaagtgtaccacatctgtgtgactgtgctgatctacttcctccccctgctggtgattggctatgcatacaccgtagtgggaatcacactatgggccagtgagatccccggggactcctctgaccgctaccacgagcaagtctctgccaagcgcaaggtggtcaaaatgatgattgtcgtggtgtgcaccttcgccatctgctggctgcccttccacatcttcttcctcctgccctacatcaacccagatctctacctgaagaagtttatccagcaggtctacctggccatcatgtggctggccatgagctccaccatgtacaaccccatcatctactgctgcctcaatgacaggtgaggatcccaaccccatgagctctccaggggccacaagaccatctacatacacagtggccaagcggcatcctaaatgagtaaacccagctgtgagacaagagggacaagtggggactgcagctaacttatcatcacacaactcagcctggctgattatcaccatccaggaatgggagcccggagtagactgattttctttttttcttttcca

SEQ ID NO: 3 - protein, 311 aa, Homo sapiens; identical to SEQ ID NO: 1

SEQ ID NO: 4 - DNA, 1268 bp, Homo sapiens; identical to SEQ ID NO: 2

SEQ ID NO: 5 - protein, 407 aa, Homo sapiens
MDNVLPVDSDLSPNISTNTSEPNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVICVIWVLALLLAFPQGYYSTTETMPSRVVCMIEWPEHPNKIYEKVYHICVTVLIYFLPLLVIGYAYTVVGITLWASEIPGDSSDRYHEQVSAKRKVVKMMIVVVCTFAICWLPFHIFFLLPYINPDLYLKKFIQQVYLAIMWLAMSSTMYNPIIYCCLNDRFRLGFKHAFRCCPFISAGDYEGLEMKSTRYLQTQGSVYKVSRLETTISTVVGAHEEEPEDGPKATPSSLDLTSNCSSRSDSKTMTESFSFSSNVLS

SEQ ID NO: 6 - DNA, 1766 bp, Homo sapiens
aattcagagccaccgcgggcaggcgggcagtgcatccagaagcgtttatattctgagcgccagttcagctttcaaaaagagtgctgcccataaaaagccttccaccctcctgtctgctttagaaggaccctgagccccaggcgccagccacaggactctgctgcagaggggggttgtgtacagatagtaggctttacgcctagcttcgaaatggataacgtcctcccggtggactcagacctctccccaaacatctccactaacacctcggaacccaatcagttcgtgcaaccagcctggcaaattgtcctttgggcagctgcctacacggtcattgtggtgacctctgtggtgggcaacgtggtagtgatgtggatcatcttagcccacaaaagaatgaggacagtgacgaactattttctggtgaacctggccttcgcggaggcctccatggctgcattcaatacagtggtgaacttcacctatgctgtccacaacgaatggtactacggcctgttctactgcaagttccacaacttctttcccatcgccgctgtcttcgccagtatctactccatgacggctgtggcctttgataggtacatggccatcatacatcccctccagccccggctgtcagccacagccaccaaagtggtcatctgtgtcatctgggtcctggctctcctgctggccttcccccagggctactactcaaccacagagaccatgcccagcagagtcgtgtgcatgatcgaatggccagagcatccgaacaagatttatgagaaagtgtaccacatctgtgtgactgtgctgatctacttcctccccctgctggtgattggctatgcatacaccgtagtgggaatcacactatgggccagtgagatccccggggactcctctgaccgctaccacgagcaagtctctgccaagcgcaaggtggtcaaaatgatgattgtcgtggtgtgcaccttcgccatctgctggctgcccttccacatcttcttcctcctgccctacatcaacccagatctctacctgaagaagtttatccagcaggtctacctggccatcatgtggctggccatgagctccaccatgtacaaccccatcatctactgctgcctcaatgacaggttccgtctgggcttcaagcatgccttccggtgctgccccttcatcagcgccggcgactatgaggggctggaaatgaaatccacccggtatctccagacccagggcagtgtgtacaaagtcagccgcctggagaccaccatctccacagtggtgggggcccacgaggaggagccagaggacggccccaaggccacaccctcgtccctggacctgacctccaactgctcttcacgaagtgactccaagaccatgacagagagcttcagcttctcctccaatgtgctctcctaggccacagggcctttggcaggtgcagcccccactgcctttgacctgcctcccttcatgcatggaaattcccttcatctggaaccatcagaaacaccctcacactgggacttgcaaaaagggtcagtatgggttagggaaaacattccatccttgagtcaaaaaatctcaattcttccctatctttgccaccctcatgctgtgtgactcaaaccaaatcactgaactttgctgagcctgtaaaataaaaggtcggaccagcttttcctcaagagcccaatgcattccatttctggaagtgactttggctgcatgcgagtgctcatttcaggatg

SEQ ID NO: 7 - protein, 407 aa, Homo sapiens [sequence omitted]

SEQ ID NO: 8 - DNA, 1230 bp, Homo sapiens
atggataacgtcctcccggtggactcagacctctccccaaacatctccactaacacctcggaacccaatcagttcgtgcaaccagcctggcaaattgtcctttgggcagctgcttacacggtcattgtggtgacctctgtggtgggcaacgtggtagtgatgtggatcatcttagcccacaaaagaatgaggacagtgacgaactattttctggtgaacctggccttcgcggaggcctccatggctgcattcaatacagtggtgaacttcacctatgctgtccacaacgaatggtactacggcctgttctactgcaagttccacaacttcttccccatcgccgcttgcttcgccagtatctactccatgacggctgtggcctttgataggtacatggccatcatacatcccctccagccccggctgtcagccacagccaccaaagtggtcatctgtgtcatctgggtcctggctctcctgctggccttcccccagggctactactcaaccacagagaccatgcccagcagagtcgtgtgcatgatcgaatggccagagcatccgaacaagatttatgagaaagtgtaccacatctgtgtgactgtgctgatctacttcctccccctgctggtgattggctatgcatacaccatagtgggaatcacactatgggccagtgagatccccggggactcctctgaccgctaccacgagcaagtctctgccaagcgcaaggtggtcaaaatgatgattgtcgtggtgtgcaccttcgccatctgctggctgcccttccacatcttcttcctcctgccctacatcaacccagatctctacctgaagaagtttatccagcaggtctacctggccatcatgtggctggccatgagctccaccatgtacaaccccatcatctactgctgcctcaatgacaggttccgtctgggcttcaagcatgccttccggtgctgccccttcatcagcgccggcgactatgaggggctggaaatgaaatccacccggtatctccagacccagggcagtgtgtacaaagtcagccgcctggagaccaccatctccacagtggtgggggcccacgaggaggagccagaggacggccccaaggccacaccctcgtccctggacctgacctccaactgctcttcacgaagtgactccaagaccatgacagagagcttcagcttctcctccaatgtgctctcctagggatcc

SEQ ID NO: 9 - DNA, 21 bp, Artificial Sequence
  Description of Artificial Sequence Primer
gacgttatccattttggggca

SEQ ID NO: 10 - DNA, 20 bp, Artificial Sequence
  Description of Artificial Sequence Primer
gacgttatccattttggggc

SEQ ID NO: 11 - DNA, 19 bp, Artificial Sequence
  Description of Artificial Sequence Primer
gacgttatccattttgggg

SEQ ID NO: 12 - DNA, 18 bp, Artificial Sequence
  Description of Artificial Sequence Primer
gacgttatccattttggg

SEQ ID NO: 13 - DNA, 17 bp, Artificial Sequence
  Description of Artificial Sequence Primer
gacgttatccattttgg

SEQ ID NO: 14 - DNA, 16 bp, Artificial Sequence
  Description of Artificial Sequence Primer
gacgttatccattttg

SEQ ID NO: 15 - DNA, 15 bp, Artificial Sequence
  Description of Artificial Sequence Primer
gacgttatccatttt

SEQ ID NO: 16 - DNA, 20 bp, Artificial Sequence
  Description of Artificial Sequence Primer
acgttatccattttggggca

SEQ ID NO: 17 - DNA, 19 bp, Artificial Sequence
  Description of Artificial Sequence Primer
cgttatccattttggggca

SEQ ID NO: 18 - DNA, 18 bp, Artificial Sequence
  Description of Artificial Sequence Primer
gttatccattttggggca

SEQ ID NO: 19 - DNA, 17 bp, Artificial Sequence
  Description of Artificial Sequence Primer
ttatccattttggggca

SEQ ID NO: 20 - DNA, 16 bp, Artificial Sequence
  Description of Artificial Sequence Primer
tatccattttggggca

SEQ ID NO: 21 - DNA, 15 bp, Artificial Sequence
  Description of Artificial Sequence Primer
atccattttggggca

SEQ ID NO: 22 - DNA, 20 bp, Artificial Sequence
  Description of Artificial Sequence Primer
ttccacatcttcttcctcct

SEQ ID NO: 23 - DNA, 21 bp, Artificial Sequence
  Description of Artificial Sequence Primer
tgatgattgtcgtggtgtgca

SEQ ID NO: 24 - DNA, 21 bp, Artificial Sequence
  Description of Artificial Sequence Primer
gcaagtctctgccaagcgcaa

SEQ ID NO: 25 - DNA, 20 bp, Artificial Sequence
  Description of Artificial Sequence Primer
ttgatgtagggcaggaggaa

SEQ ID NO: 26 - DNA, 21 bp, Artificial Sequence
  Description of Artificial Sequence Primer
tgcacaccacgacaatcatca

SEQ ID NO: 27 - DNA, 20 bp, Artificial Sequence
  Description of Artificial Sequence Primer
catagtgtgattcccactac

SEQ ID NO: 28 - DNA, 21 bp, Artificial Sequence
  Description of Artificial Sequence Primer
atgcatagccaatcaccagca

SEQ ID NO: 29 - DNA, 20 bp, Artificial Sequence
  Description of Artificial Sequence Primer
actttggtggctgtggctga

SEQ ID NO: 30 - DNA, 20 bp, Artificial Sequence
  Description of Artificial Sequence Primer
ggatgtatgatggccatgta

SEQ ID NO: 31 - DNA, 21 bp, Artificial Sequence
  Description of Artificial Sequence Primer
catggagtagatactggcgaa

SEQ ID NO: 32 - DNA, 21 bp, Artificial Sequence
  Description of Artificial Sequence Primer
gaagaagttgtggaacttgca

SEQ ID NO: 33 - DNA, 21 bp, Artificial Sequence
  Description of Artificial Sequence Primer
gtagacctgctggataaactt

SEQ ID NO: 34 - DNA, 24 bp, Artificial Sequence
  Description of Artificial Sequence Primer
acagtagatgatggggttgtacat

SEQ ID NO: 35 - DNA, 19 bp, Artificial Sequence
  Description of Artificial Sequence Primer
gtgtacagatagtaggctt

SEQ ID NO: 36 - DNA, 20 bp, Artificial Sequence
  Description of Artificial Sequence Primer
cctcctgtctggctttagaa

SEQ ID NO: 37 - DNA, 19 bp, Artificial Sequence
  Description of Artificial Sequence Primer
aacccatactgaccctttt

SEQ ID NO: 38 - DNA, 21 bp, Artificial Sequence
  Description of Artificial Sequence Primer
caaggatggaatgttttccct

SEQ ID NO: 39 - DNA, 18 bp, Artificial Sequence
  Description of Artificial Sequence Primer
tctctacctgaagaagtt

SEQ ID NO: 40 - DNA, 21 bp, Artificial Sequence
  Description of Artificial Sequence Primer
ttcgaaatggataacgtcctc

SEQ ID NO: 41 - DNA, 20 bp, Artificial Sequence
  Description of Artificial Sequence Primer
aggaggaagaagatgtggaa

SEQ ID NO: 42 - DNA, 21 bp, Artificial Sequence
  Description of Artificial Sequence Primer
tgcacaccacgacaatcatca

SEQ ID NO: 43 - DNA, 21 bp, Artificial Sequence
  Description of Artificial Sequence Primer
ttgcgcttggcagagacttgc

SEQ ID NO: 44 - DNA, 20 bp, Artificial Sequence
  Description of Artificial Sequence Primer
ttcctcctgccctacatcaa

SEQ ID NO: 45 - DNA, 21 bp, Artificial Sequence
  Description of Artificial Sequence Primer
tgatgattgtcgtggtgtgca

SEQ ID NO: 46 - DNA, 20 bp, Artificial Sequence
  Description of Artificial Sequence Primer
gtagtgggaatcacactatg

SEQ ID NO: 47 - DNA, 21 bp, Artificial Sequence
  Description of Artificial Sequence Primer
tgctggtgattggctatgcat

SEQ ID NO: 48 - DNA, 20 bp, Artificial Sequence
  Description of Artificial Sequence Primer
tcagccacagccaccaaagt

SEQ ID NO: 49 - DNA, 20 bp, Artificial Sequence
  Description of Artificial Sequence Primer
tacatggccatcatacatcc

SEQ ID NO: 50 - DNA, 21 bp, Artificial Sequence
  Description of Artificial Sequence Primer
ttcgccagtatctactccatg

SEQ ID NO: 51 - DNA, 21 bp, Artificial Sequence
  Description of Artificial Sequence Primer
tgcaagttccacaacttcttc

SEQ ID NO: 52 - DNA, 21 bp, Artificial Sequence
  Description of Artificial Sequence Primer
aagtttatccagcaggtctac

SEQ ID NO: 53 - DNA, 21 bp, Artificial Sequence
  Description of Artificial Sequence Primer
atgtacaaccccatcatctac

SEQ ID NO: 54 - DNA, 19 bp, Artificial Sequence
  Description of Artificial Sequence Primer
aagcctactatctgtacac

SEQ ID NO: 55 - DNA, 20 bp, Artificial Sequence
  Description of Artificial Sequence Primer
ttctaaagccagacaggagg

SEQ ID NO: 56 - DNA, 19 bp, Artificial Sequence
  Description of Artificial Sequence Primer
aaaagggtcagtatgggtt

SEQ ID NO: 57 - DNA, 21 bp, Artificial Sequence
  Description of Artificial Sequence Primer
agggaaaacattccatccttg

SEQ ID NO: 58 - DNA, 18 bp, Artificial Sequence
  Description of Artificial Sequence Primer
aacttcttcaggtagaga

SEQ ID NO: 59 - DNA, 21 bp, Artificial Sequence
  Description of Artificial Sequence Primer
gaggacgttatccatttcgaa